(19) United States
(12) Patent Application Publication
(10) Pub. No.: US 2007/0036809 A1
(43) Pub. Date: Feb. 15, 2007

(54) Tolerance-induced targeted antibody production

(76) Inventors: Josef Michl, Little Neck, NY (US); Stefan M. Bradu, Rego Park, NY (US)

Correspondence Address: DILWORTH & BARRESE, LLP, 333 EARLE OVINGTON BLVD., UNIONDALE, NY, 11553, US

(21) Appl. No.: 10/542,586

(22) PCT Filed: Jan. 29, 2004
(86) PCT No.: PCT/US04/02562

§ 371 Date: Oct. 18, 2005

Related U.S. Application Data
(60) Provisional application No. 60/443,703, filed on Jan. 29, 2003.

(51) Int. Cl. A61K 39/00; C12P 21/04

(52) U.S. Cl. 424/185.1; 424/277.1; 435/70.21

(57) ABSTRACT

The present invention provides methods for directing the immune response of an animal towards immunologically weak or rare antigens such as tumor antigens. The methods combine subtractive immunization with hyperimmunization and result in the controlled or directed production of target-specific antibodies, helper T cells (CD4⁺-T lymphocytes) and cytotoxic T cells (CD8⁺-T lymphocytes). Also provided by the present invention are untransformed and transformed cell lines, and growth media necessary to grow the untransformed cell line in a differentiated state. Monoclonal antibodies which react with different neoplastic cell lines and hybridomas producing such antibodies are also provided.

### BACKGROUND OF THE INVENTION

1. Field of the Invention

The present invention relates to methods for re-directing the immune response of an animal. In particular, the present invention relates to directing the immune response of an animal towards immunologically weak or rare antigens such as tumor antigens. The methods combine subtractive immunization with hyperimmunization and result in the controlled or directed production of target-specific antibodies, helper T cells (CD4⁺-T lymphocytes) and cytotoxic T cells (CD8⁺-T lymphocytes). Resultant antibodies are especially useful in diagnostic and therapeutic applications.

2. Description of the Related Art

For more than two decades mAbs have been used as powerful means for the identification of antigens present on a large variety of cells from mammalian, avian, and amphibian tissues, from plants, parasites, bacteria and viruses as well as synthetic antigens. Since the pioneering studies of K. Landsteiner in the early half of the last century, antibodies have been known to distinguish between two virtually identical proteins by their ability to specifically recognize (react with) minute differences (epitopes) in a protein's primary, secondary, and/or tertiary structure. Thus, a single amino acid change in a protein, as it may happen upon introduction of a single point mutation into the gene coding for the particular protein, can be recognized by antibodies present on the surface of B lymphocytes leading to the immune cells' proliferation into plasma cells and the secretion of antigen (epitope)-specific antibodies. As an example, antibodies are produced in diabetics injected with pig insulin; pig insulin is distinct from human insulin by only one amino acid.

The development of the hybridoma fusion procedure by Köhler and Milstein, (1975) Nature 256: 495-497, enabled the search for and the identification of antibodies carrying these refined recognition specificities, the maintenance of the producing plasma cells in permanent culture and, thus, the industrial production of the mAbs with desirable specificities. Consequently, the number of mAbs used for the delivery of diagnostic and, more recently, of therapeutic drugs and their use as therapeutics has been growing.

While the fusion procedure has become a well controlled routine methodology, the process of immunizing the (animal) donor of the immune splenocytes with a complex mixture of antigens such as intact cells, in most instances, remained a purely empirical procedure (the “standard” immunization procedure). It is therefore not surprising, that there is little predictability as to the presence and frequency of the (desired) antigen-specific antibody secreting plasma cells in the spleen of such an animal. The use of a “standard” immunization often results in the identification of only one or so hybridoma secreting a nab with desired specificity. Frequently, no mAb-secreting hybridoma of interest can be identified. Even if mAbs of apparently desired specificity are found, testing of many of the generated mAbs has demonstrated that the respective antigen(s), in most instances, is present in more cells than those of the target organ and that were used as the antigen in the immunization procedure. Clearly, these results considerably restrict the mAb's usefulness as an organ- or cell-specific vehicle in vivo.

Methodologies presently used in the production of target-specific mAbs include induction of specific immunologic tolerance. In this procedure, an immune response to immunodominant antigens is suppressed by: (a) introduction of neonatal tolerance, (b) the repeated administration of low doses of antigen, (c) the administration of immunosuppressive agents immediately before or after or during a single injection of a first set of antigens and the induction of the primary immune response (Many et al., Clin. Exptl. Immunol., 1970, 6: 87-99; Hanai et al., Cancer Res., 1986, 46:4438-4443; Middelton et al., Fed. Proc., 1984, 39:926; Golumbiski et al., Anal. Biochem. 1986, 154:373; Matthew et al., 1987, J. Immunol. Meth., 100:73-82; Pytowski et al., J. Exp. Med., 1988, 167:421; Williams et al., Biotechnique, 1992, 12:842-847; Brooks et al., J. Cell Biol., 1993, 122:1351-1359). These methods however, are still hampered by problems. For example, frequently tumor-specific antigens (TSAs) and tumor-associated antigens (TAAs) are derived by slight modifications (see above) of molecules already existing on the untransformed parent cell, and may, therefore, not be recognized within the sea of other, immunodominant antigens presented. In addition, TSAs/TAAs are presented in such low numbers that no or only a passing immune response is generated in the host.

To make full use of a mAb's potential discriminatory specificity as a targeting vehicle for a diagnostic or therapeutic purpose, the manipulation of an immunized animal's response is highly desirable so that two main objectives are achieved. First, the B lymphocyte response and, consequently, antibody production should be overwhelmingly directed towards cell and/or organ-specific antigen(s). In addition, at the time of fusion the greatest possible numbers of those plasma cells that produce the desired antibody(-ies) should have migrated to and be present in the spleen of the immunized donor animal. While the first objective should result in the proliferation of only those B lymphocytes that respond to the antigen of interest, the second objective, through the considerable enrichment of highly selected (with respect to antibody specificity) plasma cells in large numbers in the spleen, leads to a significant higher frequency of fusion between such a (desired) plasma cell(s) and myeloma cell(s). The present invention achieves both objectives and results in not only a much larger number of hybridomas growing in vitro but also a predictable higher frequency of hybridomas secreting mAbs with precisely the desired antigen-specificity.

### SUMMARY OF THE INVENTION

The present invention provides a method for redirecting the immune response of an animal towards immunologically weak or rare antigens. The method comprises the steps of: (a) administering to the animal a first set of antigens and allowing a first and secondary immune response; (b) administering to the animal an immunosuppressant which inhibits growth of rapidly proliferating immune cells; (c) administering to the animal a second set of antigens which is similar or related to, but distinct from, the first set of antigens; and (d) administering booster injections of the second set of antigens sufficient to raise the antibody titer to the second set of antigens and to cause increased immigration of plasma cells secreting antibodies to the second set of antigens into the spleen of the animal.

In another aspect of the invention, there is provided a method of producing monoclonal antibodies which react specifically with immunologically weak or rare antigens. The method comprises the steps of: (a) administering to an animal a first set of antigens and allowing a first and secondary immune response; (b) administering to the animal an immunosuppressant which inhibits growth of rapidly proliferating immune cells; (c) administering to the animal a second set of antigens which is similar or related to, but distinct from, the first set of antigens; (d) administering booster injections of the second set of antigens sufficient to raise the antibody titer to the second set of antigens and to cause increased immigration of plasma cells secreting antibodies to the second set of antigens into the spleen of the animal; (e) isolating splenocytes from the animal; and (f) fusing the isolated splenocytes with myeloma cells or transformed cells capable of replicating indefinitely in culture to yield hybridomas which secrete the monoclonal antibodies that react specifically with the immunologically weak or rare antigens. Preferably, the immunosuppressant is cyclophosphamide. In a preferred embodiment, the first set of antigens comprises untransformed cells while the second set of antigens comprises cells derived therefrom which are neoplastically transformed. For example, the first set of antigens may comprise BMRPA1 (BMPRA.430) cells and the second set of antigens may comprise BMRPA1.NNK cells. As used herein, “BMRPA1” cells and “BMRPA.430” cells are synonymous. In another example, the first set of antigens may comprise BMRPA1 (BMPRA.430) cells and the second set of antigens may comprise TUC3 (BMRPA1.K-ras^{Val12}) cells. An example of a second set of antigens are tumor associated antigens or tumor specific antigens. An example of a cancer associated antigen is a pancreatic cancer associated antigen.

In another aspect of the invention, there are provided monoclonal antibodies produced by the methods described above.

A culture medium capable of maintaining BMRPA1 cells in a differentiated state is also provided by the present invention. The culture medium comprises: about 0.02 M glutamine, about 0.01 to about 0.1M HEPES-Buffer, bovine insulin dissolved in acetic acid in a range of from about 0.001 to about 0.01 mg/mL acetic acid/L of medium), about 1 to about 8×10⁻⁷ M ZnSO₄, about 1 to about 8×10⁻¹⁰ M NiSO₄ 6H₂O, 5×10⁻⁷ to about 5×10⁻⁶ CuSO₄, about 5×10⁻⁷ to about 5×10⁻⁶ FeSO₄, about 5×10⁻⁶ to about 5×10⁻⁶M MnSO₄, about 5×10⁻⁷ to about 5×10⁻⁶ M (NH₄)₆Mn₇O₂₄, about 0.3 to about 0.7 mg/L medium Na₂SeO₃, about 1×10⁻¹⁰ to about 8×10⁻¹⁰ M SnCl₂ 2H₂O and about 5×10⁻⁴ to about 5×10⁻⁵ M carbamyl choline, wherein said medium has a pH adjusted to a range of from about 6.8 to about 7.4.

Preferably, the medium comprises about 0.02 M glutamine, about 0.02 M HEPES-Buffer, bovine insulin dissolved in acetic acid (0.004 mg/mL acetic acid/L of medium), about 5×10⁻⁷ M ZnSO₄, abut 5×10⁻¹⁰ M NiSO₄ 6H₂O, about 5×10⁻⁸ M CuSO₄, about 5×10⁻⁶ M FeSO₄, about 5×10⁻⁹ M MnSO₄, about 5×10⁻⁷ M (NH₄)₆Mn₇O₂₄, about 0.5 mg/L medium Na₂SeO₃, about 5×10⁻¹⁰ M SnCl₂ 2H₂O and about 5×10⁻⁵M carbamyl choline, wherein said medium has a pH adjusted to about 7.3.

The present invention also provides transformed BMRPA1 (BMPRA.430) cells exposed to 1 μg NNK/ml culture medium for about sixteen hours. An example of such cells is the cell line BMRPA1.NNK. The cell line TUNNK, derived from a tumor of a mouse injected with BMRPA1.NNK cells, is also provided by the present invention.

The present invention also provides a cancer associated antigen 3D4-Ag in substantially pure form characterized by: a molecular weight of about 39.0 kD as determined by SDS-PAGE, or about 41.2 kD as determined by 2D gel electrophoresis; a pI on isoelectrofocusing of about 5.9 to about 6.9 and; detectable in BMRPA1.NNK cells, BMPRA1.TUC3 cells, BMRPA1.TUNNK cells, human pancreatic cancer cells CAPAN1 and CAPAN2, A549 human lung cancer cells, and B16 mouse melanoma cells.

An antibody having binding specificity to cancer associated antigen 3D4-Ag is also provided by the present invention. The antigen is characterized by:

a molecular weight of about 41.2 kD as determined by SDS-PAGE; a pI on isoelectofocusing of about 5.9 to about 6.9 and; is detectable in BMRPA1.NNK cells, BMPRA1.TUC3 cells, BMRPA1.TUNNK cells, human pancreatic cancer cells CAPAN1 and CAPAN2, A549 human lung cancer cells, and B16 mouse melanoma cells. The antibody may be polyclonal or monoclonal. Also provided is the monoclonal antibody mAb3D4.

In another aspect of the invention, there is provided a murine hybridoma cell line which produces a monoclonal antibody specifically immunoreactive with the antigen 3D4-Ag.

The present invention also provides a hybridoma produced by the methods described herein, which hybridoma produces an antibody which binds to antigens on the surface of untransformed cells, e.g., BMRPA1 cells, and transformed cells e.g., BMRPA1.NNK cells.

Antibodies produced by a subject hybridoma wherein such antibodies bind to transformed and untransformed cells, such as the monoclonal antibodies mAb4AB 1 and mAb2B5 are also provided.

A hybridoma produced by the methods of the present invention wherein the hybridoma produces an antibody which binds to antigens of transformed cells, e.g., BMRPA1.NNK cells, but not untransformed cells, e.g., BMRPA1 cells, is also provided.

An antibody produced by a subject hybridoma wherein such antibody binds to transformed cells, but not untransformed cells, e.g., mAb3A2 is also provided.

### BRIEF DESCRIPTION OF THE DRAWINGS

FIGS. 1A through 1D are photomicrographs showing morphological changes induced by NNK in BMRPA1 cells. (FIG. 1A) Typical epithelial cobblestone-like monolayer of untreated BMRPA1. (FIGS. 1B-1F) NNK-treated BMRPA1 cells. Sequential cell passages (p2-9) after exposure to 1 μg NNK/ml in FBS-free cRPMI for 16 h: (FIG. 1B) p2: Appearance of spindle cells in the epithelial monolayer; (FIG. 1C) p6: Round cells on top and within the strands of spindle cells; (FIG. 1D) p7: Appearance of foci (arrow) throughout the TCD and beginning of colonies (arrowhead); (FIG. 1E) p9: Compact masses of cells like the ones shown, grow from many of the colonies; (FIG. 1F) Cells isolated from the core of a colony by aspiration into a thin glass needle (“cloned”) and reseeded are spindle shaped, and maintain the ability to form foci and compact masses of cells.

FIG. 2A shows culture plates of BMRPA1 (BMRPA.430), BMRPA1.NNK, and BMRPA1.K-ras^{Val12} (TUC3) cells. Foci were observed macroscopically by Hematoxylin and Eosin (H&E) staining. FIGS. 2B through 2D are photomicrographs showing foci formation by H&E staining. BMRPA1.NNK cells form basophilic foci (FIG. 2C), similar to those observed in the cultures of transformed BMRPA1.K-ras^{Val12} (TUC3) cells (FIG. 2D). Foci are not present in BMRPA1 cells grown and stained under identical conditions (FIG. 2B).

FIG. 3 graphically depicts cell growth of BMRPA1.NNK and BMRPA1 cells at 10% FBS. Cells (5×10⁴) were plated in 60 mm TCD, and allowed to grow in cRPMI supplemented with 10% FBS. At the indicated time intervals cells in triplicate dishes were released by Trypsin-EDTA and counted. In FIG. 3: filled triangles represent BMRPA1.p48 cells; filled inverted triangles represent uncloned BMRPA1.NNK.p11 cells; and open diamonds represent cloned BMRPA1.NNK.p23. Each experiment was performed twice and the results presented are representative of both trials. For each time point the average of triplicate cell counts ±SD is given.

FIGS. 4A through 4D are results of FACS analysis to demonstrate cell growth. BrdU was added to BMRPA1.p54 (FIG. 4B), uncloned BMRPA1.NNK.p1 3 (FIG. 4C), and cloned BMRPA1.NNK.p23 cells (FIG. 4D). Cells processed identically but without BrdU were used as negative controls (FIG. 4A). Cells (5×10⁴) were plated in 60 mm TCD, and allowed to grow in cRPMI supplemented with 10% FBS. Three days later BrdU was added in fresh medium and the incorporated BrdU was detected by FACS analysis. Each experiment was performed twice and the results presented are representative for both experiments. FIG. 4E is a histogram comprising data from FACS analysis of 4A-4D. The percentages of incorporated BrdU+/−SD for each of the cell lines tested are included in the Results section.

FIG. 5 graphically depicts the effect of serum deprivation on NNK-transformed and untransformed BMRPA1 cells. BMRPA1.NNK and BMRPA1 cells were seeded at 1.5×10⁴/well into 24-well TCP, and allowed to grow in cRPMI containing 1, 5 and 10% FBS. At the indicated time intervals the relative cell growth was assessed in triplicate wells by the Crystal Violet Assay (Serrano et al., 1997). The OD₆₀₀ₙₘ values at day 1 for the NNK-transformed and untransformed BMRPA1 cells were virtually identical. The growth advantage of BMRPA1.NNK cells at only 1% FBS is clearly evident when compared to the growth of BMRPA1 cells. Each experiment was performed twice and the results presented are representative of both experiments. Each time point represents the ratio of the average of OD₆₀₀ₙₘ values from triplicate wells at the indicated time point relative to the OD₆₀₀ₙₘ reading on day 1.

FIGS. 6A and 6B are photomicrographs showing H&E Staining of Nu/Nu mice tumor sections derived from subcutaneous inoculation of (A) BMRPA1.NNK.P23 cells and (B) BMRPA1.K-ras.

FIG. 7A graphically depicts efficient cyclophosphamide elimination of antibody responses to antigens expressed by untransformed cells as measured by Cell-EIA. Strong immunosuppression to BMRPA1 antigens was observed in mice immunized 3 times with BMRPA1 cells (also designated herein as BMRP.430 cells) followed by cyclophosphamide [circles, 3 immunizations (31) BMPRA430 cells (430)+Cy], and reinjected once with the same cells [squares, 3I(430)+Cy+I(430)], respectively, as compared to mice immunized 4 times with BMRPA1 cells only [triangles; 4I(430)]. Relative antibody titers were measured in duplicate, using serially diluted immune sera and Cell-EIA on BMRPA1 (BMRP.430) cells.

FIG. 7B are two photomicrographs showing immunohistochemistry on rat pancreas, confirming immunosuppression by cyclophosphamide. The sera obtained after 4 straight immunizations with BMRPA1 cells strongly stained rat pancreatic cells in situ (left). The absence of staining by sera from mice immunized three times, followed by Cy, and reimmunized with BMRPA1 cells confirms the efficiency of the cyclophosphamide-induced suppression of the immune response to BMRPA1 cells.

FIG. 7C graphically depicts that hyperimmunization with BMRPA1.NNK cells (also designated herein as BMRPA.430.NNK cells) increases antibody production. The additional 5 immunizations (5I) with BMRPA1.NNK cells in the days preceding hybridoma fusion further increased the Ab titer obtained with the standard protocol of 3I with BMRPA1.NNK cells following the cyclophosphamide immunosuppression. Cell-EIA on BMRPA1.NNK cells was done with sera after 3I(430)+Cy+3I(BMRPA1.NNK (squares) and 3I (430)+Cy+8I(BMRPA1.NNK) (circles), respectively, and with preimmune control serum (triangles). Optical density (OD 490 nm) readings of duplicate wells were averaged ±SD to measure antibody titers after the rapid hyperimmunization with the additional 5 injections of BMRPA1.NNK cells (total eight injections after cyclophosphamide treatment).

FIGS. 8A-8J are photomicrographs showing hybridoma supernatant 3C4 recognizes an Ag located on the cell surface of two independently transformed cell lines. Cells were released by EDTA, and intact, live cells on ice were reacted sequentially with 3C4 supernatant and FITC-GαM IgG. Cells were washed and mounted on glass slides and photographed under Visible (FIGS. 8A, 8C, 8E, 8G, and 8I) and UV light (FIGS. 8B, 8D, 8F, 8H, and 8J). The linear ring-like staining pattern observed with 3C4 on transformed BMRPA1.NNK (FIG. 8D) and BMRPA1.Kras^{val12} (FIG. 8F) cells, and the absence of any staining in BMRPA1 cells (FIG. 8H) indicates that 3C4 recognizes a cell-surface transformation associated antigen. FIG. 8B shows strong staining of BMRPA1.NNK cells is observed with pre-fusion sera from mice hyperimmunized with BMRPA1.NNK cells (positive control). FIG. 8J shows staining of transformed BMRPA1Kras^{val12}TUC3 processed with unreactive spent hybridoma supernatant and FITC-GαM IgG is not observed (specificity control).

FIGS. 9A through 9F are photomicrographs showing that 3D4 recognizes an intracellular antigen in BMPRA1.NNK cells that is absent from untransformed rat pancreatic cells. Immuno-cytochemical staining using mAb 3D4 or immune sera, followed by detection with HRP GαM-IgG and the HRP reaction substrate diaminobenzidine (DAB) was performed on fixed, Triton X-100 (1%) permeabilized cell lines (FIGS. 9C-9F) and frozen sections of rat pancreas (FIGS. 9A and 9B). Samples used for FIGS. 9A, 9C, and 9E were processed with mAb 3D4; samples in FIGS. 9B, 9D, and 9F were processed with sera from mice directly immunized with BMRAP1.NNK cells. Staining was observed in permeabilized BMRPA1.NNK cells (FIG. 9E) but not in permeabilized untransformed BMRPA1 cells (FIG. 9C), nor in permeabilized normal rat pancreatic tissue cells (FIG. 9A). As expected, sera from mice directly immunized with BMRPA1.NNK cells reveals extensive cross reactivity with normal pancreatic tissue (B), BMRPA1 (D), and BMRPA1.NNK cells (FIG. 10F).

FIG. 10 is a Western blot showing identification of the 3D4 antigen as an approximately 39 kD antigen in transformed BMRPA1 cells. Equal protein amounts from the respective cell lysates (30 μg) separated on 10% SDS-PAGE gels were transferred to nitrocellulose, followed by sequential incubation with mAb3D4 and HRP-Ga M IgG. The location of the Ag-Ab complex was then visualized by enhanced ECL and exposure to X-omat film: Lane 1, BMRPA1 cells; Lane 2, BMRPA1.NNK cells; Lane 3, BMRPA1.K-ras^{val12} cells. In Lane 4, spent P3U-1 myeloma medium was substituted for mAb3D4 during the immunoblotting of BMRPA1.NNK cell lysate (specificity control).

FIG. 11 is a Western blot showing identification of 3D4-Ag presence in CAPAN-1, but not in normal ductal and acinar human pancreatic cells. Western blot analysis was performed as described in FIG. 10, except that 20 μg of protein from the respective cell lysates were separated on 12% SDS-PAGE gels. Lane 1, BMRPA1.K-ras^{val12} cells (negative control, no in mAb3D4); lane 2, BMRPA1.K-ras^{val12} cells; lane 3, ARIP cells; lane 4, human pancreatic acinar tissue; lane 5, human pancreatic ductal tissue; lane 6, CAPAN-1 cells; lane 7, MIA PaCa-2 cells.

FIG. 12 is a Western blot showing identification of 3D4-Ag expression in cell lines derived from human lung cancer and mouse melanoma. Western blot analysis was performed as described in FIG. 11, except: Lane 1, human lung cancer A549 cells; lane 2, human colon carcinoma CaCO-2 cells; lane 3, human cervical carcinoma HeLa cells; lane 4, human embryonic kidney 293 cells; lane 5, human white blood cells (WBC); lane 6, mouse fibroblast L929 cells; lane 7, mouse melanoma B16 cells; lane 8, human lung cancer A549 cells exposed to spent P3U-1 myeloma medium (specificity control).

FIGS. 13A, B and C illustrate characterization of rat 3D4-Ag by 2D polypeptide separation 2D isoelectric focusing/Duracryl gel electrophoretic separation of 100 μg of polypeptides from total cell lysates, followed by Silver staining of BMRPA1 (FIG. 13A) and BMRPA1.NNK (FIG. 13B). The separated polypeptides from unstained gels run in parallel with the silver stained gels were transferred to a nitrocellulose membrane. Western blot analysis (FIG. 13D) of the membrane revealed that the rat 3D4-Ag has three charge isoforms (pIs of 6.24+/−0.25, 6.3+/−0.20, 6.5+/−0.25), and established a MW of 41.2 kD in BMRPA1.NNK cells. The nitrocellulose membrane was stained with either Amido Black or RevPro to reveal the location of 3D4-Ag in relationship to major proteins whose expression pattern was recognizable in silver-stained gels. The rat 3D4-Ag was found at the same location in 3 separate experiments (FIG. 13C, arrowheads).

### DETAILED DESCRIPTION OF THE INVENTION

The present invention relates to redirecting the immune response of an animal towards immunologically weak or rare antigens. In accordance with the present invention, there are provided methods for producing large numbers of target-specific mAbs against (i) virtually any antigenic epitope(s) by which two otherwise homologous protein antigen(s) may differ, for example, as the consequence of a single point mutation, or against (ii) any antigen that is weakly immunogenic or present in low frequency within a mixture of complex antigens. The resulting antibodies may be used in diagnosing and treating various conditions in an animal, especially a human. In addition, the present invention provides target-specific helper T cells (CD4⁺-T lymphocytes) and cytotoxic T cells (CD8⁺-T lymphocytes).

In accordance with the present invention, an immunosuppressant is administered after the complete immunization of the host with a first set of antigens, i.e., after the first and secondary immune response is completed. This results in the: (i) suppression/elimination not only of the early (primary) responding B cell clones (as in other procedures using immunosuppressive agents) but also of those B cell clones that will respond to the minor immunogens present in the initial complex antigen mixture or to immunogens that are present in lower frequency only during the secondary immune response, i.e. after the second and/or third boost; (ii) elimination of responding/proliferating B cell clones that underwent class switching and have generated memory cells which upon encountering new antigen (second & third boost) are likely to produce high affinity antibodies to any of the immunogens present in the complex antigen mixture; (iii) elimination of proliferating helper CD4⁺T_{H} lymphocytes that respond to the presentation by AP (dendritic cells>>macrophages) of processed antigens from the complex antigen mixture. Thus, the removal of these TH lymphocytes after the initial recognition of some of the antigens in the mixture by the relevant B cells will remove the help that the proliferating B cells require for class switching, for the production of higher affinity and long-lasting antibodies, and for the generation of specific memory B lymphocytes. In addition, there is (iv) generation of a long-lasting (>4 months) immunosuppression towards the initial complex antigen mixture.

Thus, the methods of the present invention are different from existing methods in that the present invention further employs a rapid sequence of immunization and hyperimmunization with the second set of desired antigen(s) in native and denatured form, and subsequent to immunization with and tolerization to the first set of antigen(s). This results in: (i) a significant rise of the antibody titer to the second set of antigens during the time period of continued suppression of the animal's response to the antigens that were present in the first complex antigen mixture; (ii) an increased immigration into the spleen of the host animal of plasma cells secreting high affinity antibody/-ies specific for the second set of antigens. Thus, it can be expected that the ratio of plasma cells in the spleen of the host animal increases in favor of those specific for the second set of antigens versus other specificities. Consequently, during hybridoma fusion there will be an increased presence within the splenocytes of the number of plasma cells producing higher affinity antibodies specific for the second set of antigens and that will fuse with the myeloma cells. This improves the chance to identify hybridomas secreting antibodies specific for the unique antigenic determinants present in the second set of antigens. In addition, there is also (iii) the production of monoclonal antibodies (mAb) to both native and denatured forms of the molecules in the second set of antigens.

In addition to the generation of a long-lasting tolerance against a first set of antigens as induced by the repeated treatment with an immunosuppressant of the post-secondary immune response, the subsequent rapid hyperimmunization of the selectively immunodeficient host animal with a related but also distinct second set of antigens leads to a strong albeit restricted, i.e., targeted immune response and antibody production to any novel antigen(s) and antigenic epitope. The continued presence of high levels of the second set of antigens in the hyperimmunized host animal exert force on the responding B cells to proliferate in large numbers, to move through class switching, and to select for plasma cells that produce higher affinity antibodies that are reactive with the native and/or denatured forms of the unique antigenic determinants within the second set of antigens. The presence at higher frequency of these plasma cells within the splenocytes of the host animal selected for subsequent hybridoma fusion significantly increases the frequency of hybridomas secreting mAbs of the desired specificity/-ies. Taken together, the methods of the present invention, therefore, constitute a major advantage over the use of standard immunization procedures in producing mAbs to select antigenic determinants within a complex mixture of antigens.

Thus the present invention provides a method for producing a target-specific monoclonal antibody comprising the following steps. First, an animal is immunized with a first set of antigens, and boosted sufficiently for complete immunization so that a first and secondary immune response is completed. Next, an immunosuppressant which inhibits growth of rapidly proliferating immune cells, including clones of B lymphocytes and T lymphocytes (cytotoxic/suppressor cells, helper cells), is administered to the immunized animal. The immunosuppressed animals are then immunized with a second set of antigens (in native and denatured form) related to but distinct from the first set of antigens, and sufficiently boosted thereafter. A hyperimmunization protocol follows, with the animal receiving within a short period of time, additional boosters of the second set of antigens. Splenocytes are isolated from the animal and fused with myeloma cells or transformed cells capable of replicating indefinitely in culture, to yield hybridomas. Resulting hybridomas may be cultured and resulting colonies screened for the production of the desired monoclonal antibody. Antibody producing colonies are grown either in Z vivo or in vitro in order to produce larger amounts of the desired antibody.

An immunosuppressant for use in the methods of the present invention should be one that inhibits growth of rapidly proliferating immune cells including clones of B lymphocytes and T lymphocytes. Especially useful compounds include those of the classes alkylating agents, antimetabolites, and natural products. Examples of such compounds include but are not limited to, cyclosporine A, mycophenolate, mofetil, azathioprine, tacrolimus, leflunomide, mycophenolic acid, melphalan, chlorambucil, methotrexate, fluoruracil, vincristine, busulfan, and cyclophosphamide. Preferably, cyclophosphamide is used as the immunosuppressant in the methods of the present invention.

Antigens for use in the methods of the present invention can encompass any material effective in stimulating an immune response in a vertebrate organism. Thus for example, an antigen may be an infectious agent such as a bacterium or virus. An antigen for use in the present invention may also comprise an isolated protein, peptide or fragment thereof. Such a protein, peptide or fragment thereof, may be isolated from an infectious agent or other live source, be chemically synthesized or recombinantly produced. In addition, a small molecule such as a hapten may function as an antigen for use in the methods of the present invention. Preferably, the antigen is a surface protein of an infectious agent or neoplastic cell. Even more preferably, the antigen is a tumor-associated antigen (TAA) or tumor-specific antigen (TSA). TAAs have been identified for a number of tumors, including melanoma, breast adenocarcinoma, prostatic adenocarcinoma, esophageal cancer, lymphoma and many others. See Shawler et al. (1997) Advances in Pharmacology 40:309-337, Academic Press.

Thus, an antigen for use in the methods of the present invention may comprise virtually any antigenic determinant (epitope) (i) by which two otherwise homologous protein antigen(s) may differ, for example, as the consequence of a single point mutation, or (ii) any antigen that is weakly immunogenic or present in low frequency within a mixture of complex antigens. Two protein antigens are homologous if they possess a variation in amino acid sequence by any combination of additions, deletions, or substitutions but otherwise possess the same functional property or are fragments derived from proteins sharing the same functional property. In order to generate monoclonal antibodies specific to an antigenic determinant (epitope) by which two otherwise homologous protein antigen(s) may differ, or specific to an antigen that is weakly immunogenic or present in low frequency within a mixture of complex antigens, two sets of related but distinct antigens are employed.

The two related but distinct sets of antigens may be obtained through several means. For example, cells may be isolated from a first tissue source and may be used as a first set of antigens while cells from a second tissue source from the same organism may be used as a second set of antigens. Examples of cells which may serve as sources of first and second sets of antigens include cells from different pancreatic tissue such as duct cells, central acinar cells, acinar cells, and islet cells. In another example, different layers of brain tissue may be used as many types of brain cells are derived from precursor cells. In still another example, thyroid cells and parathyroid cells may serve as a first and second set of antigens. Adrenal gland tissue is also made of different cell types which may serve as a first and second sets of antigens. In yet another example, ovarian cancer-specific antigens may be isolated using cells isolated from an undiseased ovary from a subject as primary antigen and cells isolated from a diseased ovary from the same subject as a secondary antigen.

The methods of the present invention are especially useful in generating mAb against TSAs and TAAs, which as described above, are often derived by slight modification of molecules already existing on the untransformed parent cell. Such TSAs and TAAs may therefore be unrecognizable among the myriad of other immunodominant antigens presented. The TSAs/TAAs may also be presented in such low numbers that only a passing immune response or no immune response is generated in the host. Thus for example, with respect to TSAs and TAAs, an untransformed parent cell line and a transformed neoplastic cell line may be used as the first and second set of similar or related, yet distinct antigens. Neoplastic transformation is known to occur via K-ras oncogenic mutations and methylation of the p16 tumor suppressor gene promoter leading to loss of P16 protein expression (Belinsky et al. 1998). Thus, cells may be transformed with a vector such as a plasmid comprising a K-ras oncogenic mutation or a plasmid comprising a nucleotide sequence which can inactivate the p16 tumor suppressor gene. In addition, exposure of cells to various nitrosamines including 4-(methyl-nitrosamino)-1-(3-pyridyl)-1 butanone (NNK), has been shown to result in the formation of DNA and protein adducts, DNA strand breaks, and gene mutations (Curphey et al., 1987; Van Benthem, et al., 1994; Staretz et al., 1995; Hecht, 1996;). The nicotine-derived NNK and its metabolite 4-(methyl-nitosamino)-1-(3-pyridil)-1-butanol (NNAL), are useful for producing pancreatic tumors in lab animals (Hoffman, D., et al. 1994, J. Tox., and Env. Health 41:1-52) and are especially useful for inducing neoplastic transformation of pancreatic cells. NNK exposure time for pancreatic cells may range from any time from about six hours to about sixty hours. A preferred range of exposure is from about twelve hours to about twenty four hours. An exposure time of about sixteen hours is especially preferred.

There is a wide array of carcinogenic substances known to transform normal cells into neoplastic cells. In accordance with the present invention, cells may be exposed to various compounds in order to produce neoplastic cells. Examples of such compounds include but are not limited to nitrosamines such as NNK and other classes such as alkylating agents, aralkylating agents, arylalkylating agents, arylaminating agents and polycyclic aromatic hydrocarbons. These compounds and the use of such compounds for generating neoplastic cells are described in numerous publications such as Yuspa, S. H., Shields, P. G., “Etiology of cancer: chemical factors” in Cancer, Principles and Practice of Oncology, Devita Jr., V. T., Hellman, S., Rosenberg, S. A. (eds.), Lippincott Williams and Wilkens, Philadelphia, 6^{th} ed., pp. 179-193, the disclosure of which is hereby incorporated by reference as if fully set forth. The foregoing carcinogenic substances are not meant to be inclusive but merely exemplary. Many different carcinogenic substances may be used to produce neoplastic cells for generating TAAs or TSAs useful for practicing the methods of the present invention.

Tumorous tissue or cells taken directly from an animal source often contain a mixture of normal and cancer cells as well as connective tissues and proteases. Therefore, transformed cell lines are preferably used as an antigen or source of antigen in the methods of the present invention. An untransformed, parental cell line may serve as a first set of antigens while a cell line derived therefrom, which has been neoplastically transformed, may serve as the second set of related (similar) yet distinct antigens.

In accordance with the methods of the present invention, an immunosubtractive hyperimmunization protocol (“ISHIP”) described above, has been used to produce targeted antibodies. The general method, also denoted “tolerance-induced targeted antibody production” is described more specifically below.

At the start of the protocol (day 0), animals are bled for preimmune serum. The animals, preferably mice, are immunized with a first set of antigens referred to as complex antigen profile “A”. Preferably, the first set of antigens is administered by intraperitoneal (ip) or subcutaneous (sc) injection. In addition, a mixture of live and fixed cells is preferably used as the first set of antigens, i.e., complex antigen profile “A”. For example, BMPRA.430 cells, described infra, may be used as complex antigen profile “A”. Compounds and formulations of such compounds, which may be used to fix cells are well known in the art and include e.g., formaldehyde, glutaldehyde, and paraformaldehyde. Paraformaldehyde is preferably used to fix cells in the methods of the present invention.

The animals are then boosted twice with a mixture of live and fixed complex antigen profile “A”. At days 12-15, a first booster injection is given by e.g., intraperitoneal injection of live/fixed complex antigen profile “A” at 50% the cell number or protein concentration used in the injection on day 0. At days 18-21, a second booster injection is given and comprised of live/fixed complex antigen profile “A” at the same concentration as on day 0. Preferably, the second booster is by subcutaneous administration.

The animals may then be weighed to determine the baseline weight, which can be later used to determine the effect of the immunosuppressant (discussed in greater detail below). At approximately 4-24 hours after the second booster injection, animals may be bled in order to obtain immune serum, and the serum may be tested for antibodies against antigen profile “A.”

Over the next five days (days 23-26), the animals may be weighed each day and then administered an immunosuppressant, such as cyclophosphamide at 60 mg/kg BW diluted in sterile physiological saline solution. Preferably, administration of cyclophosphamide is by intraperitoneal (ip) injection. A typical schedule of treatment is as follows. At 24 hours after the second booster injection, animals are weighed and cyclophosphamide administered intraperitoneally at 60 mg/kg BW. 48 hours after the second booster injection animals are weighed again and cyclophosphamide administered intraperitoneally at 60 mg/kg BW. 72 hours after the second booster injection, animals are again weighed and administered cyclophosphamide at 60 mg/kg BW. 96 hours after the second booster injection there is a weighing of animals and cyclophosphamide is administered at 60 mg/kg BW. Finally, at 120 hours after the second booster injection animals are again weighed and cyclophosphamide administered at 60 mg/kg BW. Preferably, administration of cyclophosphamide is by i.p.

An observed weight loss of 2-10% in cyclophosphamide-treated animals is a general indicator of the drug's effect, since treatment with this drug has the effect of decreasing the animals' food and fluid intake. After the last injection of cyclophosphamide, animals may be weighed daily for a period of about 10-12 days. At the end of such time period, the animals will have regained their pretreatment weight. Indicia of effectiveness of immunosuppressant drugs other than cyclophosphamide may of course be used when appropriate. For example, a blood sample may be obtained and platelet and white blood cell (WBC) levels determined, which levels would be expected to be depressed after immunosuppressant drug treatment.

Blood is then collected from the immunized animals (days 33-36), and antibody titer in the immune serum established against antigen profile A (e.g. BMRPA.430 cells) and against a second set of closely related, yet distinct antigens. It is this set of antigens, against which the animals are being directed to make an immune response i.e. modified antigen profile “A+” or “A+na”. Preferably, the second set of antigens comprise transformed cells, such as e.g., the transformed cell line designated BMRPA.430.NNK or BMRPA1.NNK (described infra). The blood samples are tested with preimmune serum and the serum taken 5 hours after the second boost, i.e., immediately before the first cyclophosphamide injection. Expected results are outlined below in Table 1:

TABLE 1
Test Antigens

| | Ag profile “A” | Ag profile “A+” or “A + na” |
|---|---|---|
| Pre-immune sera: | 0 | 0 |
| Ser. days 18-21: | +++ | ++/+++ |
| Ser. days 33-36: | 0 | 0 |

The immunosuppressed mice are then immunized by intraperitoneal or subcutaneous injection on day 37 with antigen profile “A+” or “A+na” cells (e.g. a mixture of live (50%) and paraformaldehyde-fixed (50%) cells, here BMRPA.430.NNK cells).

A first booster of the antigen profile “A+” or “A+na” (i.e. live/fixed cell mixture) is administered by intraperitoneal injection on days 49-52 at 50% the cell number of the injection at day 37. The second booster of the antigen profile “A+” or “A+na” (i.e. live/fixed cell mixture) is by intraperitoneal injection on days 55-58 at 75% of the cell number of the injection at day 37.

Serum is then collected for testing and the following hyperimmunization protocol is undertaken. At day 60-63, a booster of antigen profile “A+” or “A+na” is administered at the dosage level used on day 37. At days 62-65, a fourth booster injection is administered as a repeat of the injection of days 60-63. Preferably, administration is by s.c. injection. On days 64-67, a fifth booster injection is given at 1.5× the amount of antigen profile “A+” or “A+na” injected on day 37. At days 66-69, a sixth booster injection is administered which is a repeat of the injection of days 64-67. These last two boosters are administered preferably by i.p. injection.

At days 68-71, a seventh booster injection is administered which is a repeat of the injection of days 64-67. At days 70-73 (Day of Fusion —2 days), an eighth booster injection which is a repeat of the injection of days 64-67 is administered.

On days 71-74, sera are obtained from the immunized animals and individually tested for the presence of antibodies against antigen profiles “A+” and “A+na”, as well as “A” and antigens to which the animals had not been exposed, i.e., a group of irrelevant antigens or cells (Ir—Ag).

Expected results are outlined below in Table 2:

TABLE 2
Tested Ag profiles

| | “A” | “A+” or “A + na” | “Ir—Ag” |
|---|---|---|---|
| Serum, days 33-36: | 0 | 0 | 0/+ |
| Serum, days 55-58: | 0 | ++ | 0 |
| Serum, days 71-74: | 0/+ | ++++ | 0/+ |

On days 72-75, splenocytes are isolated for fusion from one or more mice as defined by the sera antibody titer in tests on days 71-74, and sera are collected for additional testing for the presence of antibodies against antigen profiles “A+” and “A+na”, as well as “A” and “Ir—Ag”.

As described above, splenocytes obtained from an immunized animal are fused with myeloma cells or transformed cells capable of replicating indefinitely in culture to yield a hybridoma. Methods of producing hybridomas are well known in the art and include for example, those procedures described in Köhler and Milstein (1975) and Pytowski (1988), the disclosures of which are incorporated by reference herein as if fully set forth. Individual hybridoma cells are cloned and the clones are tested for production of antibodies to “A+” or “A+na”. For example, hybridoma supernatants may be screened for antigen-specific antibody reactivities. Once a hybridoma cell line producing antibodies that react with antigens “A+” or “A+na” is identified, the cells may be frozen and stored ensuring long-term supply. Such cell lines may be subsequently thawed when more antibody is required, ensuring long-term supply.

Subject antibodies find different uses in diagnostics and therapeutics. With respect to diagnostic uses, an antibody produced in accordance with the present invention may be used as a tool to immunologically define cross reactivity with an antigen. For example, antibodies produced in accordance with the present invention may react to different antigenic determinants (epitopes) on the same antigen and are useful as diagnostics or controls. In addition, a subject antibody which is specific for a type of tumor cell, is useful for indicating changes occurring in such tumor cells and may be useful for monitoring a patient's treatment. For example, as tumor cells die, antigens are shed into the blood and serum and a subject antibody is useful in determining such changes occurring in tumor cells. In addition, antibodies produced in accordance with the present invention which react with a specific antigen e.g., a tumor specific antigen, are useful as therapeutics, either administered alone or conjugated to a cytotoxic drug.

The following examples further illustrate the invention.

### EXAMPLE 1

### Development of Cell Line BMRPA.430.NNK (BMRPA1.NNK) through Neoplastic Transformation of Pancreatic Cell Line BMRPA.430

Materials: 1640 RPMI medium, penicillin-streptomycin stock solution (10,000 U/10,000 mg/mL)(P/S), N-2-hydroxyethylpiperazine-N′-2-ethanesulfonic acid (HEPES) buffer, 0.2% Trypsin with 2 mM Ethylene diamine tetraacetic acid (Trypsin-EDTA), and Trypan blue were all from GIBCO (New York). Fetal bovine serum (FBS) was from Atlanta Biologicals (Atlanta, Ga.). Dulbecco's Phosphate Buffered Saline without Ca²⁺ and Mg²⁺ (PBS), and all trace elements for the complete medium were purchased from Sigma Chemical Company (ST. Louis, Mo.). Tissue culture flasks (TCFs) were from Falcon-Becton Dickinson (Mountain View, C.A.), tissue culture dishes (TCDs) were obtained from Corning (Corning, N.Y.), 24-well tissue culture plates (TCP), and 96-well TCP were from Costar (Cambridge, Mass.). Filters (0.22, 0.45 μm) were from Nalgene (Rochester, N.Y.).

Preparation of complex RPMI (cRPMI) cell culture medium: cRPMI was prepared with RPMI, glutamine (0.02M), HEPES-Buffer (0.02M), bovine insulin dissolved in acetic acid (0.02 mg/mL acetic acid/L of medium), hydrocortisone (0.1 μg/mL), trace elements that included ZnSO₄ (5×10⁷M), NiSO₄ 6H₂O (5×10⁻¹⁰ M), CuSO₄ (10⁻⁸ M), FeSO₄ (10⁻⁹ M), MnSO₄ (10⁻⁹ M), (NH₄)₆Mn₇O₂₄ (10⁻⁷ M), Na₂SeO₃ (0.5 mg/L medium), SnCl₂ 2H₂O(5×10⁻¹⁰ M) and carbamyl choline (10⁻⁵ M), and the pH was adjusted to 7.3. The medium was sterile filtered.

Cells and Culture: BMRPA.430 (BMRPA1) is a spontaneously immortalized cell line established from normal rat pancreas (Bao et al., 1994). TUC3 (BMRPA1.K-ras^{Val12}) are BMRPA1 cells transformed by transfection with a plasmid containing activated human K-ras with oncogenic mutation at codon 12 (Gly->Val)(Dr. M. Perucho, California Institute for Biological Research, La Jolla). All cell lines are maintained routinely in cRPMI (10% FBS) in a 95% air-5% CO₂ incubator (Form a Scientific) at 37° C. The cells are passaged by trypsin-EDTA. Cells are stored frozen in a mixture made of 50% spent medium and 50% freezing medium containing fresh cRPMI with 10% FBS and 10% DMSO. Cell viability was assessed by trypan blue exclusion.

NNK Exposures: All preparations of the carcinogen-containing media were made in a separate laboratory within a NCI-designed and certified chemical hood using prescribed protective measures. NNK (American Health Foundation, N.Y.) was prepared as a stock solution of 10 mg NNK in PBS and added to FBS-free cRPMI to make final concentrations of 100, 50, 10, 5, and 1 μg/ml. BMRPA1 cells at passage 36 (p36) were seeded at 10⁵/60 mm TCDs and allowed to grow for 6 d. At this time the medium was removed, and the cells were washed 2× with prewarmed (37° C.), FBS-free cRPMI before they were treated with FBS-free cRPMI (4 ml/TCD) containing the different concentrations of NNK. A 6th set of TCDs containing BMRPA1 cells was incubated in FBS-free cRPMI without NNK and was used as controls. The eight TCDs used for each of the six sets of different culture conditions were returned to the 37° C. and 95% air-5% CO₂ incubator. After 16 h, the NNK-containing medium was removed from all TCDs and the cells were washed 3× with PBS followed by addition of fresh cRPMI-10% FBS (4 ml/TCD), and the incubation continued. Control cultures without NNK were processed in parallel. The cells were fed every 2 d by replacing ½ of the spent medium with fresh cRPMI-10% FBS. At full confluency the cells were collected from all TCDs, the cells in each group were pooled, and passaged at 2×10⁴ into fresh TCDs.

Isolation of Colonies: To facilitate the picking of cells from individual colonies of transformed cells, cell cultures containing colonies were reseeded at 10⁵ cells/100 mm TCDs, and grown for 7 d. The narrow ends of sterile Pasteur pipettes were flamed, rapidly stretched and broken at their thinnest point to create a finely drown-out glass needle narrow enough to pick up only the core of a cell-rich colony. Only the NNK treated cells contained cell-rich, ball-like colonies. The center cores of 8 prominent colonies were picked, and each core consisting of ˜80-200 tightly packed cells was placed into a separate well each of a 24-well dish. The cells of 4 colonies thus transferred survived and were expanded.

Cell Growth Assays: To measure cell growth at 10% FBS, cells were seeded at 5×10⁴ cells/60 mm TCD containing 4 ml of cRPMI-10% FBS. Every 3 d, triplicate TCDs were removed for each cell line under study, the cells were released with trypsin-EDTA, and counted in the presence of trypan blue. To assess the effect of cRPMI containing reduced FBS concentrations on cell growth, equal numbers (1.5×10⁴ cells/ml/well) of NNK-treated and untreated BMRPA1 cells were seeded in triplicate wells of 24 well TCDs. The cells were allowed to adhere overnight in cRPMI 10% FBS, washed with PBS, and reincubated with cRPMI containing the indicated % FBS. Cell growth was evaluated by a modification of the crystal violet relative proliferation assay (Serrano, 1997). Briefly, the cells were washed with PBS, fixed in 10% buffered formalin followed by rinsing with distilled water. The cells were then stained with 0.1% Crystal Violet for 30 min at room temperature (RT), washed with dH₂O, and dried. The cell-associated dye was extracted with 1 ml 10% acetic acid, aliquots were diluted 1:2 with dH₂O, and transferred to 96-well microtiter plates for OD₆₀₀ₙₘ measurements. The cell growth was calculated relative to the OD₆₀₀ₙₘ values read at 24 h.

BrdU Incorporation: Cells (5×10⁴) were plated in 60 mm TCD, and allowed to grow in cRPMI-10% FBS. Three days later, fresh medium with BrdU (10 uM) was added for 3 h, the cells were washed, released with Trypsin-EDTA, and the incorporated BrdU was detected with an FITC conjugated anti-BrdU antibody (Becton Dickinson) by FACS analysis as suggested by manufacturer (Becton Dickinson). Briefly, 10⁶ trypsin-EDTA released cells were washed twice in PBS-1% BSA, fixed in 70% ethanol for 30 min, and resuspended in RNAase A(0.1 mg/mL) for 30 min at 37° C. After washing the cells, their DNA was denatured with 2N HCl/Triton X-100 for 30 min, and neutralized with 0.1 M Na₂B₄O₇.10H₂O, pH 8.5. The cells were then washed in PBS-1% BSA with 0.5% Tween 20, and resuspended in 50 uL of 0.5% Tween in PBS-1% BSA solution with 20 uL of FITC-AntiBrdU antibody. After 45 min at 37° C., the cells were washed, resuspended in 1 mL of Na Citrate buffer containing Propidium Iodide (0.005 mg/mL) and RNAase A (0.1 mg/mL). Fluorescent activated cell sorting or flow cytometry (FACS) analysis to detect the incorporated BrdU and PI staining was performed by using a FACScan analyzer from Becton Dickinson Co. equipped with an Argon ion laser using excitation wavelength of 488 nm. Data analysis was performed using the LYSYS II program.

Independent samples t-test was used to show statistically significant (p<0.05) differences in the percentage of the untransformed and transformed cells that incorporate BrdU. The DNA index was calculated as previously described (Barlogie et al., 1983; Alanen et al., 1990) from the DNA histogram as the ratio of the PI staining measurement for the G0/G1 peak in the transformed cells examined divided by the PI staining measurement for the G0/G1 peak in the untransformed BMMRPA1 cells.

Anchorage Independent Growth: Aliquots of 4 ml of 0.5% agar-medium mixture (agar was autoclaved in 64 mL H₂O, cooled in a water bath to 50° C., and added to 15 mL 5× cRPMT, 19 mL FBS and 1 mL P/S) were poured into 25 cm²TCFs and allowed to harden overnight at 4° C. Prior to plating the cells, the flasks were placed in the CO₂-Air incubator for up to 5 h at 37° C. to facilitate equilibration of pH and temperature. Cells were collected by Trypsin-EDTA, 0.1 mL of cell suspension (40000/mL cells in cRPMI) was dispersed carefully over the agar surface of each flask and the cultures were returned to the 37° C. incubator with 95% O₂-5% CO₂. After 24 h, the agar-coated TCFs were inverted to allow drainage of excess medium. The cultures were examined microscopically after 9 d and 14 d for growth of colonies using a Zeiss inverted microscope.

Tumorigenicity in Nu/Nu mice: Nu/Nu mice (7 wks of age) were obtained from Harlan Laboratories (Indianapolis, Ind.). The cells used for injection were released by Trypsin-EDTA, washed in cRPMI, and resuspended in PBS at 10⁸ cells/mL. Each mouse tested was injected subcutaneously (s.c.) with 0.1 ml of this cell suspension. The animals were inspected for tumor development daily during the first 4 weeks, and thereafter at weekly intervals. Small pieces of the tumors (1-2 mm³) were cut from the core of the tumors and placed in 4% paraformaldehyde overnight at 4° C. The tissue was then washed in PBS, and placed in 30% sucrose for another 24 h. Sections of tumor tissue frozen in Lipshaw embedding matrix (Pittsburgh, Pa.) were made with a Jung cryostat (Leica), placed on gelatin coated slides, and stored at −20° C. H&E staining was done according to standard procedures.

Establishment of the TUNNK cell line from excised Nu/Nu mice tumors.

Isolation of cells from tumors that grew from the BMRPA1.NNK cells that had been transplanted subcutaneously into Nu/Nu mice was done similar to the method described by Amsterdam, A. and Jamieson, J. D., 1974, J. Cell Biol. 63:1037-1056, with several procedural changes. The tumor-bearing Nu/Nu mice were sacrificed by CO₂ asphyxiation, placed on an ice-cooled bed, the skin over the tumor opened and the tumor rapidly removed surgically and sterilely, and placed into L-15 medium (GIBCO, Grand Island, N.Y.) on ice for immediate processing. While still in ice-cold L-15 medium, the tissue was minced into small pieces, followed by 2 cycles of enzymatic digestion and mechanical disruption. The digestion mixture in L-15 medium consisted of collagenase (1.5 mg/ml) (136 U/mg; Worthington Biochem. Corp.), Soybean trypsin inhibitor (SBTI) (0.2 mg/ml) (Sigma Chem. Comp.), and bovine serum albumin (BSA; crystallized) (2 mg/ml) (Sigma). After the first digestion cycle (25 min, 37° C.), the cells and tissue fragments were pelleted at 250×g, and washed once in ice-cold Ca⁺⁺ and Mg⁺⁺-free phosphate buffered saline (PD) containing SBTI (0.2 mg/ml), BSA (2 mg/ml), EDTA (0.002 M) and HEPES (0.02 M) (Boehringer Mannheim Biochem., Indianapolis) (S-Buffer). The cells were pelleted again, resuspended in the digestion mixture, and subjected to the second digestion cycle (50 min, 37° C.). While still in the digestion mixture, the remaining cell clumps were broken apart by repeated pipetting of the cell suspension using pipettes and syringes with needles of decreasing sizes. The cell suspension was then sheared sequentially through sterile 200μ-mesh and 20μ-mesh nylon Nytex grids (Tetko Inc., Elmsford, N.Y.), washed in S-Buffer and resuspended in 2-3 ml L-15 medium, centrifuged at 50×g for 5 min at 4° C. The cell pellet was collected, washed in PBS, and resuspended in cRPMI. A sample of the fraction was processed for viable cell counting by Trypan blue (Fisher Sci.) exclusion (Michl J. et al., 1976, J. Exp. Med. 144(6), 1484-93) and for cytochemical analysis. Cells were seeded and grown in cRPMI at 10⁵ cells/35 mm well of a 6-well TCD.

Photomicroscopy: All observations and photography of cell cultures were done on a Leitz Inverted Microscope equipped with phase optics and a Leitz camera. Observations were recorded on TMX ASA100 Black and White film.

### EXAMPLE 2

### Results

Effects of NNK on BMRPA1 morphology: Repeated exposures to NNK and other nitrosamines have been observed to induce both cytotoxic and neoplastic morphological alterations in a variety of rodent and human in vitro experimental models of pancreatic cancer (Jones, 1981, Parsa, 1985, Curphey, 1987, Baskaran et al. 1994). With the purpose of determining whether such changes are induced by a single exposure to NNK and at relatively small NNK concentrations, BMRPA1 cells were exposed for one 16 hour period to serum free medium containing 100, 50, 10, 5, and 1 μg NNK/mL. As observed in previous studies with pancreatic cells, the larger concentrations of NNK resulted in cytotoxic changes consisting of poorly attached, degenerating, dying cells, and slowed cell growth, while such changes were observed considerably less in cells exposed to 5, and 1 μg NNK/mL. The degenerative changes of the treatment with 100, 50, 10 μg NNK/ml were followed within a week by the appearance of phenotypical changes indicative of neoplastic transformation such as spindle morphology and focal overcrowding. BMRPA1 cells treated with NNK at 1 μg/ml also displayed phenotypical changes characteristic of neoplastic transformation but at a slower rate, over several weeks. As suggested for other mutagens (Srivastava and Old, 1988), the changes observed at lower doses might be more likely to reflect specific, preferential molecular sites of NNK-induced lesions at doses closer to those encountered in the human environment. Furthermore, the gradual pace of these changes at 1 μg/mL allows a passage by passage study of both early and late events in the process of NNK-induced transformation. Thus, the results presented below were obtained with BMRPA1 cells exposed once for 16 h to 1 μg NNK/mL FBS-free medium.

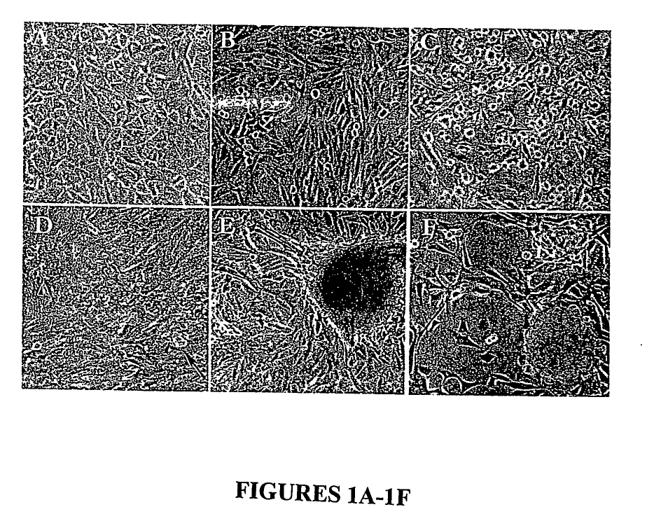

BMRPA1 cells grown continuously in culture for 35 passages were organized into a monolayer, cobblestone-like pattern typical of untransformed, contact inhibited epithelial cells (FIG. 1A). Two weeks after exposure to 1 μg NNK/ml, the BMRPA1 cells exhibited minute morphological changes: cells in a few discrete areas started losing their polygonal shape, and islands of cells consisting of spindle-shaped cells with less cytoplasm and darker nuclei started forming (FIG. 1B, p2). Beginning with passage 6 (p6) an increasing number of round cells on top and within the strands of densely packed spindle cells were observable (P6-8), suggesting loss of contact inhibition (FIG. 1C).

Island-like areas of crowded cells (foci) became prominent by p7 (FIG. 1D, arrow head), and ball-like aggregations of cells began to form on the top of these foci as colonies (p7-11). The first clearly distinguishable colonies were seen at p8-9, about 3 months after NNK exposure. Initially the colonies were small (FIG. 1D, arrow) and only few, but they were present in all 6 TCFs in which the NNK-treated BMRPA1 cells were passaged. The colonies continued to grow horizontally and vertically as compact masses (FIG. 1E) with much reduced adhesiveness, e.g., crowded cells could be easily separated by trypsinization and repeated pipetting, indicating that such cultures likely comprise neoplastic cells. The rapid disruption by trypsinization of such colonies is in direct contrast to untransformed BMRP430 (BMRPA1) cells. The control BMRPA1 cells that had been continuously cultured in parallel after 16 h exposure to FBS-free cRPMI without NNK did not show any changes and were indistinguishable from the original monolayer of BMRPA1 cells.

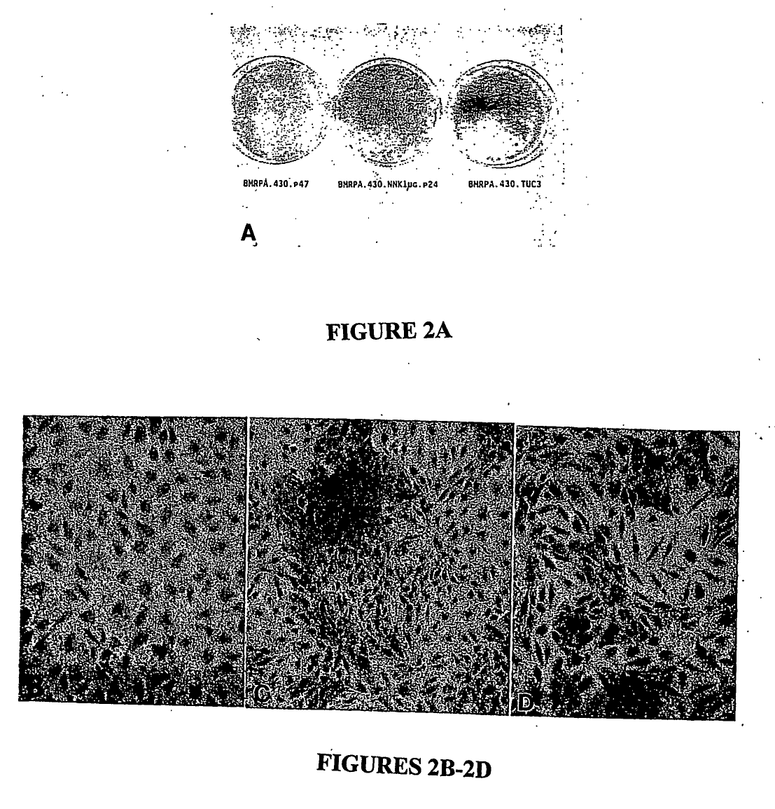

To facilitate the study of phenotypical and molecular characteristics of colony-forming cells, the cores of several colonies were isolated with a finely drown out glass needle, and each isolate of 80-200 cells was grown separately as cell lines referred to as “cloned BMRPA1.NNK”. The isolated cells displayed a spindle to triangular shape and were often multi-nucleated with different sized nuclei containing one or more prominent nucleoli. When reseeded in new flasks, these cells maintained the ability to form foci and colonies (FIG. 1F). Interestingly, the NNK-induced phenotypic changes seen in the NNK-transformed BMRPA1 are similar to but less pronounced than those observed during the transformation of BMRPA1 by human oncogenic K-ras^{val12}. The NNK-induced basophilic foci that can be easily observed macroscopically (FIG. 2A) and microscopically (FIG. 2C) after H&E staining are also similar to those formed by BMRPA1 cells transformed by transfection with oncogenic K-ras^{val12} (FIGS. 2A and 2D). In contrast, neither foci nor colonies were formed during the growth of untreated BMRPA1 cells (FIGS. 2A and B). The morphological changes induced by NNK in BMRPA1 cells are also similar to well-established characteristics of other transformed cells cultured in vitro: spindly and triangular cell shape at low cell density, rounded with halo-like appearance at high cell density, and loss of contact inhibition as indicated by growth in foci and on top of their neighboring cells (Chung, 1986).

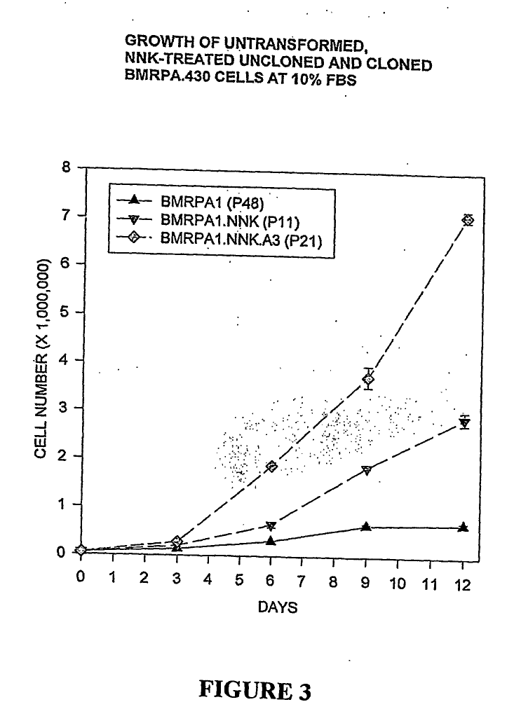

NNK-Induced Hyperproliferation: The long-term, permanent effects of NNK on the proliferation of BMRPA1 cells was initially assessed by comparing the cell growth of NNK-treated and untreated cells cultured in complex medium (cRPMI supplemented with 10% FBS. The BMRPA1, uncloned NNK-treated BMRPA1 cells, and “cloned” BMRPA.1NNK cells, i.e., isolated cells produced as described above, this example, were seeded at equal density in TCDs. At predetermined days the cells in TCDs were released by Trypsin-EDTA, collected, and counted in the presence of trypan blue. As shown in FIG. 3, untreated BMRPA1 cells at passage 46 (p46) reached a plateau around day 9 indicative of contact inhibited growth. In contrast, the NNK-treated cells grown in parallel for eleven passages after the NNK treatment showed faster growth during the first 9 d (FIG. 3), and later the growth slowed down possibly due the continued presence of untransformed BMRPA1 cells that were unaffected by NNK. The cloned BMRPA.1NNK cells isolated from the core of the NNK-induced colonies (FIG. 1F) continued to grow unimpeded throughout the 12 days of culture at a considerably faster rate than the untreated BMRPA1 cells resulting in very dense overcrowding.

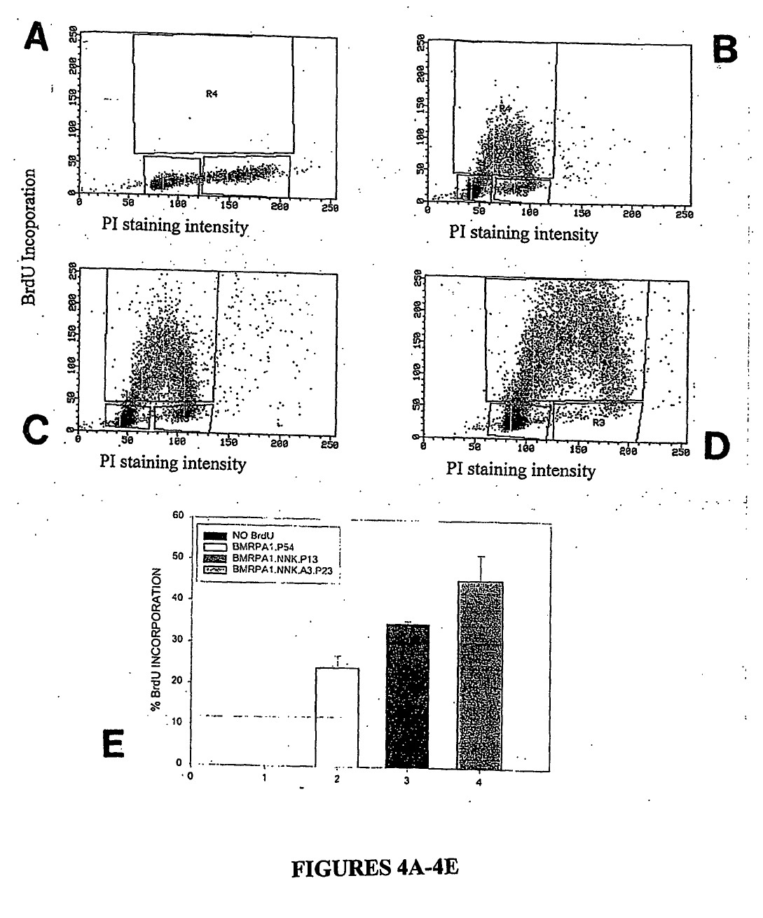

Since the cell growth curves were able to reveal significant growth differences between the NNK-treated and untreated BMRPA1 cells only at high cell densities where contact inhibited growth and cell death might contribute significantly to the observed cell growth, the increased intrinsic capacity of the NNK-treated cells to proliferate at low cell density was further assessed by measuring the ability of these cells to incorporate BrdU. The measurement of BrdU incorporation in RNAase treated cells is routinely used to assess DNA synthesis during the S phase of proliferating cells (Alberts B., Johnson, A., Lewis, J., Raff, M., Roberts, K., Walter, P., 2002, Molecular Biology of the Cell, Garland Science, Taylor and Francis, 4th ed., NY). The results obtained by FACS analysis of the BrdU incorporation in the untransformed BMRPA1.p58, transformed uncloned BMRPA.NNK.p11, and transformed cloned BMRPA.NNK.p23 cells offer further evidence that the NNK treatment resulted in permanent hyperproliferative changes in BMRPA1 (FIGS. 4A-4E). These observations provide experimental evidence that NNK is able to transform BMRPA1 cells by inducing both a focal loss of contact inhibition and hyperproliferation.

Effect of Serum Deprivation on untransformed and NNK-transformed BMRPA1 cells: One frequently cited characteristic of transformed cells is their selective growth advantage at low concentrations of growth factors and serum, conditions that poorly support the growth of primary and untransformed cells (Chung, 1986; Friess, et al., 1996; Katz and McCormick 1997). To establish the serum dependency of the untransformed and NNK-transformed BMRPA1 cells, the cells were transferred into cRPMI medium supplemented with 1%, 5%, and 10% FBS, seeded at equal cell numbers into the wells of 24-well TCPs, and grown for 12 days. A crystal violet assay was used to assess the relative cell growth (Serrano, 1997). This assay provides a significant advantage over the counting of cells released by Trypsin-EDTA because it eliminates the loss of cells (incomplete release and cell death) that occurs due to strong cell adhesion to TCDs at low serum concentrations.

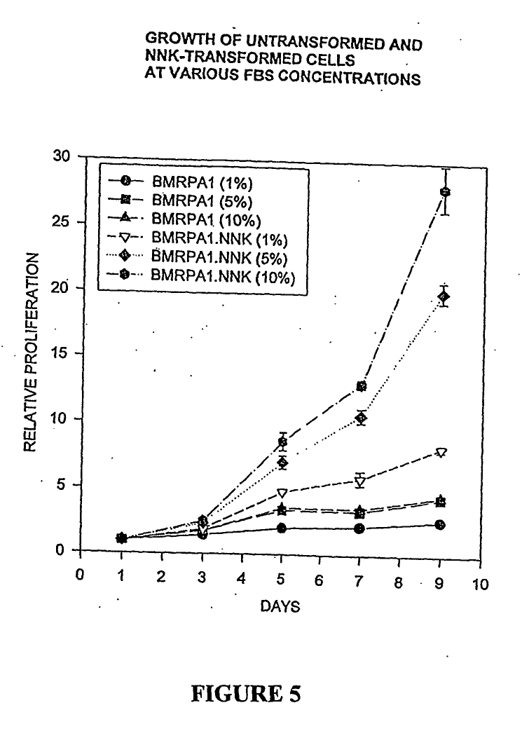

As it can be seen in FIG. 5, transformed BMRPA.1NNK cells have a selective growth advantage over untreated cells at all the FBS concentrations examined. Even in cRPMI medium containing 1% FBS the NNK-transformed cells grow better than untreated BMRPA1 cells cultured in cRPMI with 10%. The observed ability of BMRPA1.NNK cells to sustain cell growth in severely serum-deprived conditions provides further support for the transformation of BMRPA1 cells by exposure to NNK.

Anchorage-independent Cell Growth:

The malignant transformation of many cells has been shown to result in a newly acquired capability to grow on agar, under anchorage independent conditions (Chung, 1986). The ability of the cloned BMRPA1.NNK and untreated BMRPA1 cells to grow on agar was examined by dispersing cells at low density onto soft agar (see Example 1). The ability of these cells to form colonies over a 14 d period is presented in Table 3.

TABLE 3
Anchorage independent colony formation on agar
by control BMRPA1 and NNK-treated BMRPA1 cells.

| | Days after | # of colonies* formed | | |
|---|---|---|---|---|
| Cells | seeding | <50 cells | >50 cells | Total |
| BMRPA1 | 9 | 0 | 0 | 0 |
| | 14 | 0 | 0 | 0 |
| BMRPA1.NNK | 9 | 14 | 15.8 ± 2.5 | 17.3 ± 5.2 |

*using an ocular counting grid the colonies were counted in a series of 30 sequential 1 mm² fields. Average counts of colonies from 5 TCFs +/− SEM are presented.

Confirming previous observations (Bao et al., 1994), the BMRPA1 cells were unable to grow on agar and died. In contrast, BMRPA1.NNK cells showed a strong capacity to grow and form colonies. In fact, about 1 in 4 BMRPA1.NNK cells seeded formed colonies larger than 50 cells. The growth on agar is indicative of neoplastic transformation
Tumorigenicity in Nu/Nu Mice:

Cells growing on agar often have the ability to grow as tumors in Nu/Nu mice (Shin et al., 1975; Colburn et al., 1978). The ability of cells to grow in Nu/Nu mice as tumors is believed to be a strong indication of malignant transformation (Chung, 1986). Consequently, 10⁷ cloned, live BMRPA1.NNK cells were injected subcutaneously (s.c.) in the posterior flank region of Nu/Nu mice. Another group of mice was injected s.c. under similar conditions with untransformed BMRPA1 cells. A third group of Nu/Nu mice was injected with BMRPA1.K-ras^{val12} cells for positive control purposes, since these cells have been previously shown to form tumors in Nu/Nu mice.

TABLE 4
Tumorigenicity of BMRPA1.NNK cells in Nu/Nu mice.

| | # of mice with tumor/ | # of mice with metastasis/ |
|---|---|---|
| Cells | # of mice tested | # of mice tested |
| BMRPA1 | 0/5 | 0/5 |
| BMRPA1.NNK | 3/6 | 1/6 |
| BMRPA1.K-ras^{val12} | 5/5 | 1/5 |

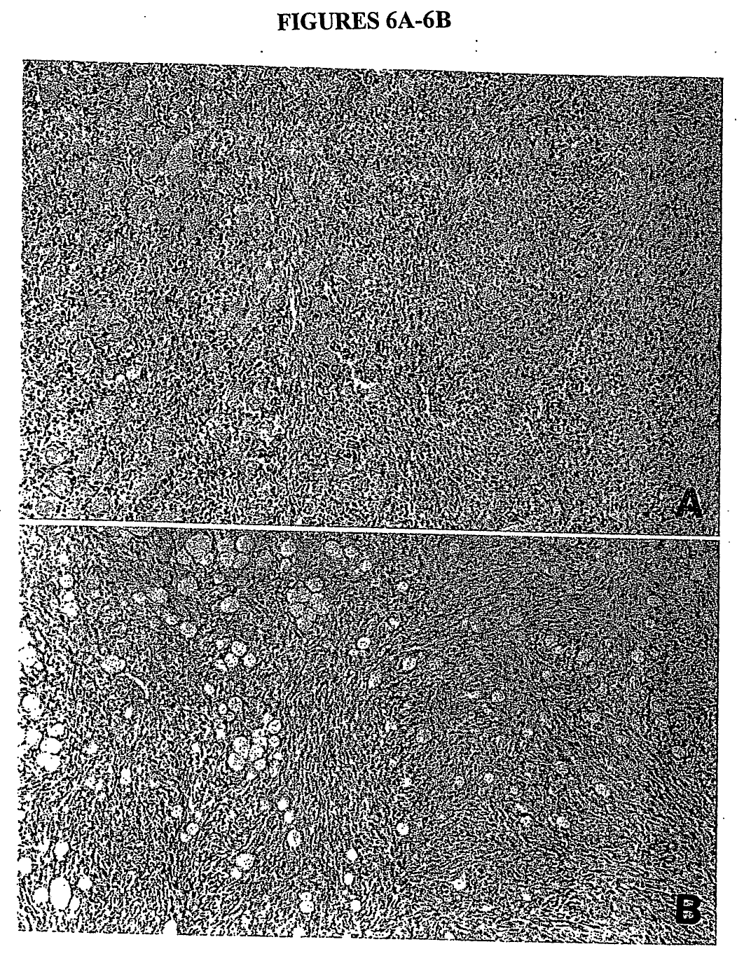

BMRPA1 cells were unable to form tumors in the 5 Nu/Nu mice injected, while BMRPA1.K-ras^{val12} formed rapidly growing nodules (<0.5 cm) that became tumors (>1 cm) within 4 wks after inoculation. Distinctly different was the course of tumor formation in the Nu/Nu mice injected with cloned BMRPA1.NNK cells. Within a week after injection with cloned BMRPA1.NNK cells, nodules of 2-3 mm formed at the injection site of all six mice. The nodules disappeared in 3 of the animals within 2 months. Nevertheless, after a period of dormancy of up to 4 months, the nodules in the remaining 3 animals evolved within the next 12-16 weeks into tumors of more than 1 cm in diameter. One of these mice carrying a large tumor mass further developed ascites suggesting the presence of metastatic tumor cells. The histopathological appearance of the tumors formed by BRMPA.NNK and by the BMRPA1.K-ras cells are presented in FIGS. 6A and 6B.

A cell line named TUNNK was established from one of the tumors growing in BMPRA1.NNK injected Nu/Nu mice by a method combining mechanical disruption and collagenase digestion. TUNNK has transformed morphological features similar to the cloned BMRPA1.NNK cells injected into the Nu/Nu mouse. So far, the only prominent distinguishing phenotypical characteristic between the two is a predisposition of TUNNK to float in vitro as cell aggregates, suggesting that significant changes in the adhesion properties of the cells took place during the selective growth process in vitro. To examine whether the selective growth of the NNK-transformed cells in Nu/Nu mice resulted in further increases of the initial NNK-induced hyperproliferation, the BrdU incorporation of the TUNNK cells was also determined under conditions identical to those presented in FIG. 4. The proliferation of TUNNK was slightly less than that of the cloned BMRPA1.NNK which were initially introduced subcutaneously into the Nu/Nu mice (FIG. 4). Nevertheless, the observed ability of the NNK-transformed cells to form tumors in Nu/Nu mice showed that a single 16 h exposure to 1 μg NNK/ml affected an important, rate limiting step in the malignant transformation of BMRPA1 cells.

### EXAMPLE 3

### Use of Tolerance-Induced Antibody Production to Identify Tumor Associated Antigens

Materials And Methods:

Materials: RPMI 1640, DMEM containing 5.5 mM glucose (DMEM-G+), penicillin-streptomycin, HEPES buffer, 0.2% trypsin with 2 mM EDTA, Bovine serum albumin (BSA), Goat serum, and Trypan blue were from GIBCO (New York). Fetal bovine serum (FBS) was from Atlanta Biologicals (Atlanta, Ga.). Hypoxanthine (H), Aminopterin (A), and Thymidine (T) for selective HAT and HT media and PEG 1500 were purchased from Boehringer Mannheim (Germany). Diaminobenzidine (DAB) was from BioGenex (Dublin, Calif.). PBS and Horseradish peroxidase labeled goat anti-Mouse IgG [F(ab′)₂ HRP-GαM IgG] were obtained from Cappel Laboratories (Cochranville, Pa.). Aprotinin, pepstatin, PMSF, sodium deoxycholate, iodoacetamide, paraformaldehyde, Triton X-100, Trizma base, OPD, HRP-GαM IgG, and all trace elements for the complete medium were purchased from Sigma (ST. Louis, Mo.). Ammonium persulfate, Sodium Dodecyl Sulfate (SDS), Dithiothreitol (DTT), urea, CHAPS, low molecular weight markers, and prestained (Kaleidoscope) markers were obtained from BIORAD (Richmond, Calif.). The enhanced chemiluminescent (ECL) kit was from Amersham (Arlington Heights, Ill.). Mercaptoethanol (2-ME) and film was from Eastman Kodak (Rochester, N.Y.). Tissue culture flasks (TCF) were from Falcon (Mountain View, Calif.), tissue culture dishes (TCDs) from Corning (Corning, N.Y.), 24-well TC plates (TCPs) and 96-well TCPs were from Costar (Cambridge, Mass.). Tissue culture chambers/slides (8 chambers each) were from Miles (Naperville, Ill.).

Cells and Culture: All rat pancreatic cell lines were grown in cRPMI containing 10% FBS. The other cell lines were obtained from the American Tissue Culture Collection (ATCC), except for the rat capillary endothelial cells (E49) which were from Dr. M. DelPiano (Max Planck Institute, Dortmund, Germany). White blood cells were from healthy volunteer donors, and human pancreatic tissues (unmatched transplantation tissues) were provided by Dr. Sommers from the Organ Transplantation Division at Downstate Medical Center. Cell viability was assessed by trypan blue exclusion.

Immunosubtractive Hyperimmunization Protocol (ISHIP): A mixture of live (10⁶) and paraformaldehyde fixed and washed (10⁶) cells was used for each immunization intraperitoneally (ip). Six female Balb/c mice (age˜12 wks) (Harlan-Sprague Dawley Labs, St. Louis) were used: two mice were injected 4× during standard immunizations with BMRPA1 cells. The other four mice were similarly injected 3× with BMRPA1 cells, and 5 h after the last booster injection they were injected ip for the next 5 d with 60 μg cyclophosphamide/day/g of body weight. Two of these immunosuppressed mice were reinjected with BMRPA1 cells after the last cyclophosphamide injection. The other two immunosuppressed mice were injected weekly three more times with transformed BMRPA1.NNK cells, and a week later the mice were hyperimmunized with 5 additional injections of transformed BMRPA1.NNK cells in the 10 days preceding fusion (ISHIP mice). Sera were obtained from all mice within a week after the indicated number of immunizations.

Hybridomas and mAb Purification: Hybridomas were obtained as previously described (Köhler and Milstein, 1975; Pytowski et al., 1988) by fusion of P3U1 myeloma cells with the splenocytes from the most immunosuppressed ISHIP mouse. Hybridoma cells were cultured in 288 wells of 24-well TCPs. The hybridomas were initially grown in HAT DMEM-G+(20% FBS) medium for 10 d, followed by growth in HT containing medium for 8 d, and then in DMEM-G+(20% FBS). Hybridoma supernatants were tested 3× by Cell-Enzyme ImmunoAssay (Cell-EIA) starting 3 weeks after fusion for the presence of specific reactivities before the selection of specific mAb-containing supernatants for further analysis by imunofluorescence microscopy and immunohistochemistry was made. MAb 3D4 was purified by precipitation in 50% saturated ammonium sulfate of hybridoma supernatant, and later the precipitate was dissolved in PBS and dialyzed against PBS. MAb 3D4 was identified as a mouse IgG1 antibody and separated from the dialyzed material by Sepharose-Blue chromatography as previously described (Pytowski et al., 1988). The IgG fraction contained ˜10.5 mg protein /mL as measured by the Bradford's assay (BioRad).

Cell-Enzyme ImmunoAssay (Cell-EIA): BMRPA1 and BMRPA1.NNK cells were seeded in TCPs (96-wells) at 3×10⁴/well with 0.1 mL cRPMI-10% FBS. The cells were allowed to adhere for 24 h, air dried, and stored under vacuum at RT. The cells were then rehydrated with PBS-1% BSA, followed by addition of either hybridoma supernatants or two fold serial dilutions of mouse sera to each well for 45 min at room temperature (RT). After washing with PBS-BSA, HRP-GαMIgG (1:100 in PBS-1% BSA) was added to each well for 45 min at RT. The unbound antibodies were then washed away, and OPD substrate was added for 45 min at RT. The substrate color development was assessed at OD₄₉₀ₙₘ with a microplate reader (Bio-Rad 3550). For hybridoma supernatants, an OD₄₉₀ₙₘ, value greater than 0.20 (5× the negative control OD value obtained with unreactive serum) was considered positive.

Indirect Immunofluorescence Assay (IFA) On Intact Cells: Cells were released by incubation with 0.02 M EDTA in PBS, washed with PBS-1% BSA, and processed live at ice cold temperature for imunofluorescence analysis. The cells were incubated for 1 h in suspension with hybridoma supernatants or sera, washed (3×) in PBS-1% BSA, and exposed to FITC-GA M IgG diluted 1:40 in PBS-1% BSA. After 45 min, unbound antibodies were washed away, and the cells were examined by epifluorescence microscopy.

Immunoperoxidase Staining of Permeabilized Cells and Tissue Sections. Preparation of cells and tissues: Transformed and untransformed BMRPA1 cells were seeded at 1×10⁴ cells/0.3 mL cRPMI/chamber in Tissue Culture Chambers. Two days later, the cells were fixed in 4% paraformaldehyde in PBS overnight at 4° C. The cells were then washed twice with PBS-1% BSA and used for immunohistochemical staining. Pancreatic tissue for immunohistochemical staining was prepared from adult rats perfused with 4% paraformaldehyde in 0.1M phosphate buffer, pH 7.2. The fixed pancreas was removed from the fixed rat and stored overnight in 4% buffered paraformaldehyde at 4° C. The pancreas was then washed and placed in 30% sucrose overnight. Frozen tissue sections (10 μm) were made with a Jung cryostat (Leica), placed on gelatin-coated glass slides, stored at −20° C. The cell lines or tissue sections were then post-fixed for 1 min in 4% buffered paraformaldehyde, washed in Tris buffer (TrisB) (0.1 M, pH 7.6), and placed in Triton X-100 (0.25% in TrisB) for 15 min at RT. Then immunohistochemistry was done as previously described (Guz et al., 1995).

Western Blot Analysis of 3D4-Ag: The cell lines tested for the presence of 3D4-Ag were grown to confluence in 25 cm² TCDs, washed with ice-cold PBS, and incubated on ice with 0.5 mL RIPA lysing buffer (pH 8) consisting of 50 mM Tris-HCl, 1% NP40, 0.5% sodium deoxycholate, 0.1% SDS, 5 mM EDTA, 1 μg/mL pepstatin, 2 μg/mL aprotinin, 1 mM PMSF, and 5 mM iodoacetamide. After 30 min, the remaining cell debris was scraped into the lysing solution, and the cell lysate was centrifuged at 11,500× g for 15 min to remove insoluble debris. Cell lysates from pancreatic tissues were processed in a similar manner for the Western blot analysis, with the difference that 2 pieces of ˜2 mm³ per tissue type were homogenized in a Dounze homogenizer in 1 mL of RIPA lysing buffer at ice temperature. The protein concentration of each lysate was determined by the Bradford's assay (BioRad). The cell extracts were mixed with equal volumes of sample buffer (125 mM Tris-HCl, 2% (v/v) 2-mercaptoethanol, 2% SDS, 0.1% bromophenol blue, 20% v/v glycerol, pH 6.8). The proteins from each sample (20 μg/well) were separated by SDS-PAGE as previously described (Laemmli, 1970), and electrotransferred onto nitrocellulose membrane. After the membrane was incubated with 5% (w/v) dry milk in TBS-T for 1 h, mAb 3D4 (1:200) and the HRP-G a M IgG were added and the chemiluminescence amplified using the ECL kit as suggested by the manufacturer (Amersham). The presence of the protein of interest due to chemiluminescence in each of the samples tested was detected by exposure to X-OMAT film (Kodak).

2D Isoelectric focusing/SDS-Duracryl Gel Electrophoretic Polypeptide Separation. Untransformed and NNK-transformed cells were plated at 10⁵ cells/25 cm² TCF, fed every 3 d, and grown until the untransformed cells reached confluence. The cells in the flasks were then lysed either in RIPA buffer for Bradford's protein measurement or in a lysing buffer solution made of 0.1 g DTT, 0.4 g CHAPS, 5.4 g Urea, 500 uL Bio-lyte ampholyte, 6 mL ddH₂O, 5 mM EDTA, 1 μg/mL pepstatin, 2 ug/mL aprotinin, 1 mM PMSF, and 5 mM iodoacetamide. The cell lysates were centrifuged at 11,500×g for 15 min to remove insoluble debris. Precast first and second dimension gels and equipment from Genomic Solutions (MA) were then used. Protein (100 μg) was loaded into the first dimension (pI 3-10) which was run at 300V for 3 h, and then at 1000V for 17 h. The second dimension for each experiment was run using precast 10% SDS-Duracryl gels (Genomic Solutions, MA) at 20 mA/gel. The separated polypeptides were either rapidly transferred onto a nitrocellulose membrane under semi-dry conditions for 1 h at 1.25 mA/cm² (484 mA), or silver stained according to the manufacturer's instructions (Genomic Solutions, MA). The nitrocellulose membrane was then used for 3D4-Ag detection by Western blot analysis, and was later stained with either Rev Pro (Genomic Solutions, MA), or Amido Black (Sigma). The pH gradient of 0.5 cm sections from the first dimension gel was determined as previously described (O'Farrell, 1975). The silver staining of the 2D separated polypeptides was photographed using 100 ASA Black and White (Kodak) film.

Photomicroscopy: All observations and photography of stained cell cultures or issue samples were done with a Leitz inverted Photomicroscope equipped with a camera and phase optics, using 125 ASA Black and White, 400 ASA Ektachrome (Kodak), or 1600 ASA PROVIA (Fuji) film.

### EXAMPLE 4

### Results

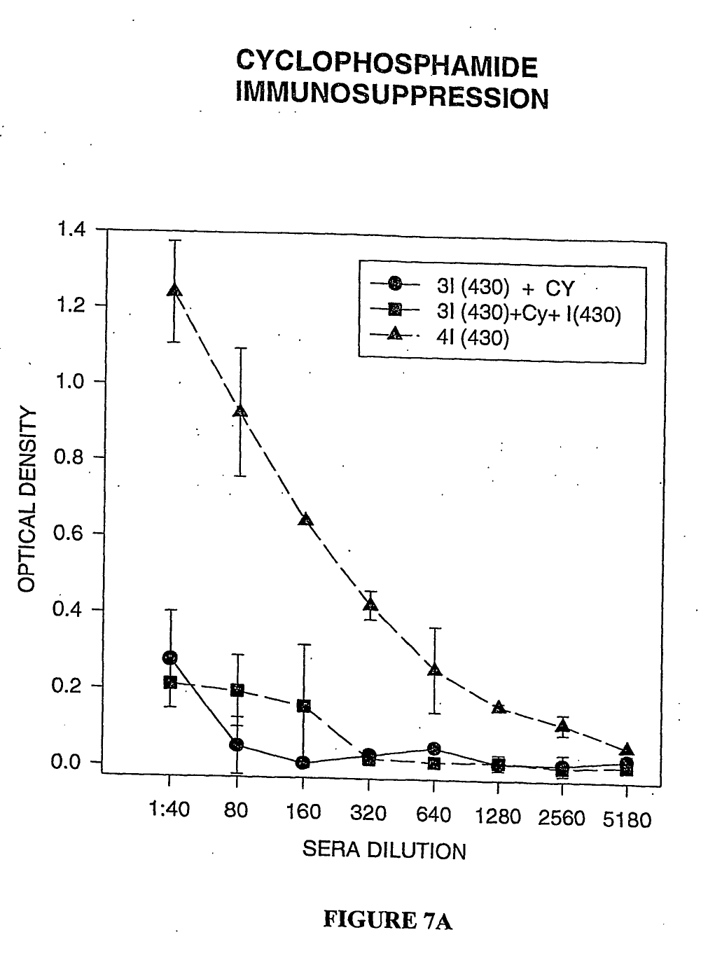

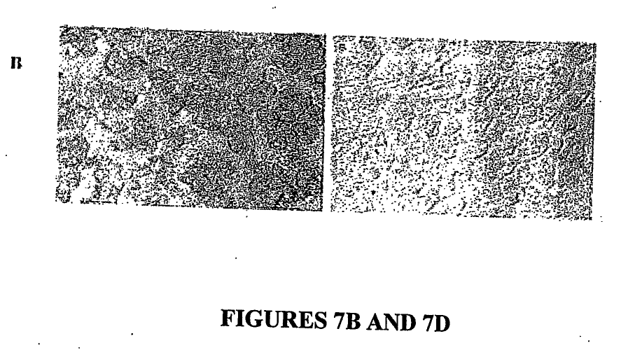

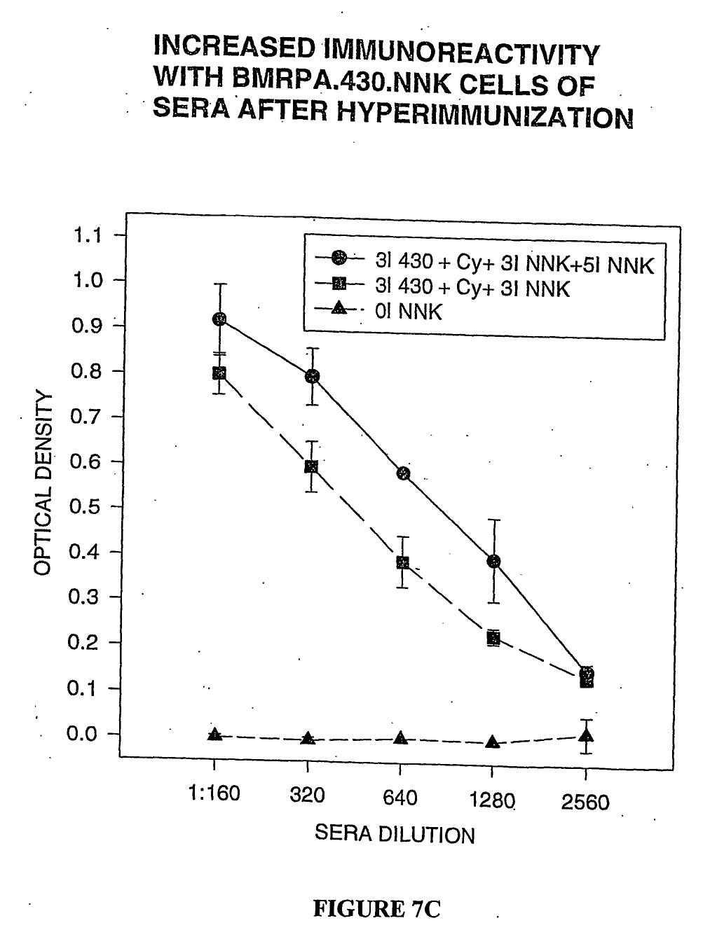

The immunosubtractive hyperimmunization protocol (ISHIP): Immunosubtractive methods developed to produce antibodies that are able to recognize differences between two closely related complex antigens take advantage of the ability of well defined doses of cyclophosphamide to preferentially kill B-cells which have been stimulated to proliferate mostly in response to the immunodominant epitopes shared by the complex Ags (Aisenberg, 1967; Aisenberg and Davis, 1968; Williams et al., 1992; Matthew and Sandrock, 1987; Pytowski et al., 1988). In the past, administration of cyclophosphamide after immunization with a large dose of Ag in the form of sheep red blood cells resulted in very efficient Ag-specific immunological tolerance, while if the drug was administered after a lower dose of Ag the specific immunological tolerance was not as efficient (Aisenberg 1967; Aisenberg and Davis, 1968; Playfair, 1969). To improve the effectiveness of cyclophosphamide in eliminating the clones of immune cells proliferating in response to Ags present on untransformed BMRPA1 cells (the “tolerogen”), an immunization protocol was designed in which 3 immunizations with BMRPA1 cells were followed by cyclophosphamide (FIG. 7). The extent of immunosuppression by cyclophosphamide was initially evaluated by Cell-EIA with sera from immunized and cyclophosphamide-treated mice on dried BMRPA1 cells. Sera collected from mice immunized 4 times i.p. with BMRPA1 cells contained considerable antibody titers for these cells (FIG. 7A). In contrast, when 3 injections of BMRPA1 cells were followed 5 h later and for the next 5 days by i.p. injections of cyclophosphaimide, strong immunosuppression was observed in all 4 mice examined. Remarkably, a booster injection with BMRPA1 cells after the cyclophosphamide treatment did not result in the recovery of the antibody titer to the tolerogen (FIG. 7A). These results were confirmed by immunohistochemistry on rat pancreatic tissue (FIG. 7B). A strong crossreactivity of sera from mice immunized with BMRPA1 cells was observed with rat pancreatic tissue (FIG. 7B, left), while the sera from BMRPA1 immunized and subsequently cyclophosphamide-treated mice showed virtually no staining of rat pancreatic tissue (FIG. 7B, right).

Cyclophosphamide at the dose used in this study has been shown in mice to preferentially kill Ag-specific proliferating B cells and T cells, but it also has additional, non-specific cytotoxic effects on spleen cells (Aisenberg, 1967; Aisenberg and Davis, 1968; Turk et al., 1972; Lagrange et al., 1974; Marinova-Mutafclieva et al., 1990; Pantel et al., 1990). Such previously described non-specific immunosuppression was reported to be present in immunosubtractive protocols at 3 to 7 wks after the cyclophosphamide treatment (Aisenberg 1967, 1968), which is the time when the transformed BMRPA1.NNK cells (novel Ag) would be introduced in the animals tolerized to the untransformed BMRPA1 cells (tolerogen). This partial state of non-specific immunosuppression can decrease the number of B-cells specific for transformation Ags present in the spleen of the animals used for fusion possibly decreasing the production of desired mAbs. Furthermore, even in classical immunizations when an animal with an intact immune system is injected with cancer cells, the transformation associated Ags were observed to have low immunogenicity (Old, 1981; Shen et al., 1994). To minimize these potential problems and to increase the number of B-cells stimulated to proliferate by tumor antigens, the immunosuppression of the secondary immune response to BMRPA1 cells by cyclophosphamide was followed by i.p. immunization with BMRPA1.NNK cells, two booster injections 10 and 16 d later, and a rapid hyperimmunization with another 5 booster injections of transformed cells in the days preceding the hybridoma fusion. Cell-EIA done on the sera collected before and after hyperimmunization from the mouse used for the hybridoma fusion showed that the rapid hyperimmunization with the 5 injections of BMRPA1.NNK cells resulted in an increase in the antibody titers to BMRPA1.NNK cells (FIG. 7C).

Detection of antigenic differences between NNK-transformed and untransformed BMRPA1 cells: Hybridoma supernatants collected from 288 wells were tested by Cell-EIA for the presence of IgG antibodies reactive with dried NNK-transformed and untransformed BMRPA1 cells. Evaluation on days 18 to 21 after fusion established that 265 (92%) of the 288 wells examined contained one or more growing hybridomas. By Cell-EIA, supernatants from 73 (or 23.5%) of the wells contained antibodies that reacted with transformed BMRPA1.NNK cells. In contrast, only 47 (or 16.3%) supernatants reacted with BMRPA1 cells, indicating that BMRPA1.NNK cells express antigens which are not expressed by the untransformed BMRPA1 cells. Moreover, all 47 hybridoma supernatants reactive with BMRPA1 cells exhibited crossreactivity with transformed BMRPA1.NNK cells.

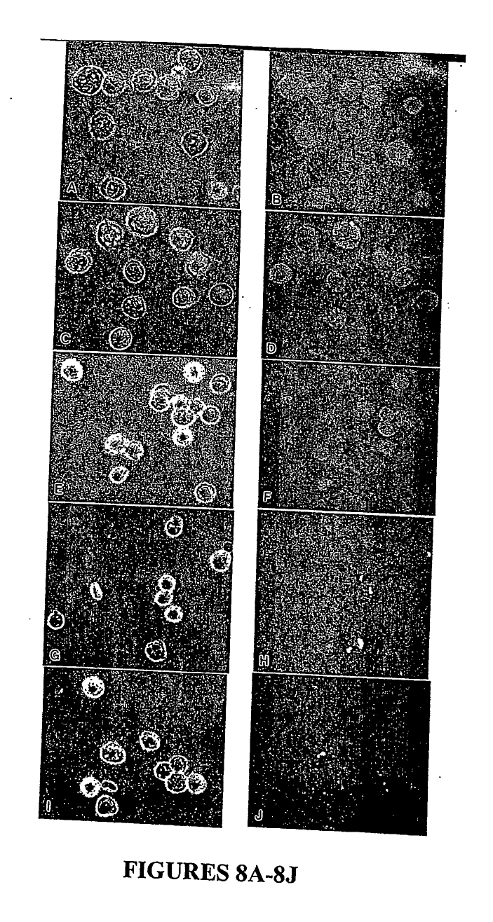

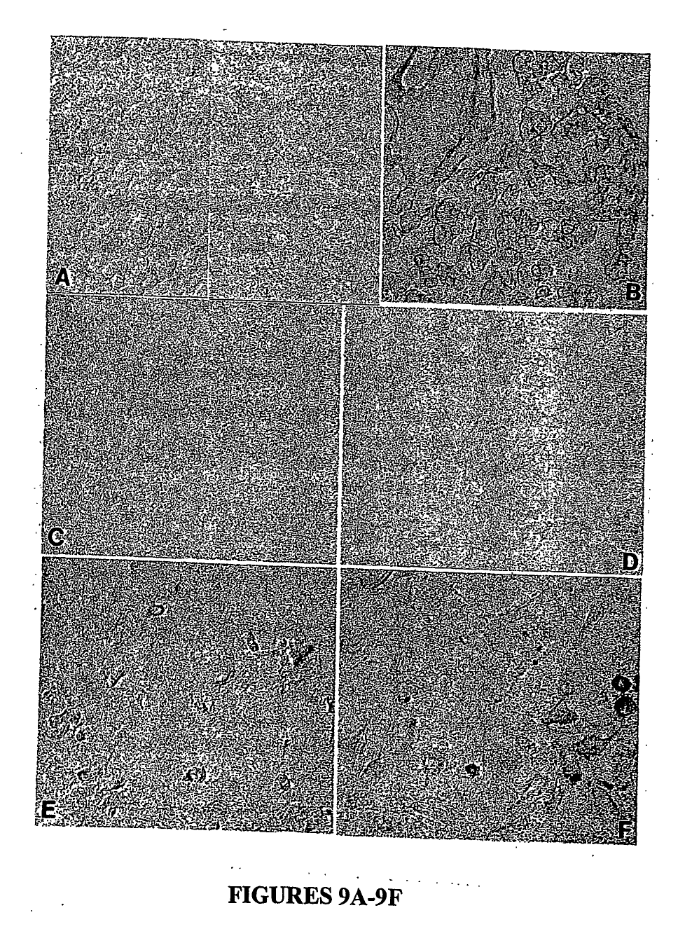

Immunoreactivity of selected hybridoma supernatants with intact untransformed and transformed BMRPA1 cells: As the Cell-EIA testing was performed on dried, broken cells, the antibodies in the supernatants could access and bind both intracellular and plasma membrane Ags. To obtain initial information regarding the cellular location of the recognized Ags, 5 hybridoma supernatants were initially selected for further testing by IFA on intact cells because by Cell-EIA these supernatants consistently showed promising strong reactivity either with only BMRPA1.NNK cells (supernatants 3A2; 3C4; 3D4), or with both BMRPA1.NNK and BMRPA1 cells (supernatants 4AB1; 2B5). As summarized in Table 5, supernatants 3C4, 4AB1, and 2B5 stained the cell surface of intact cells in agreement with the Cell-EIA results. Remarkably, 3C4 stained BMRPA1.NNK (FIG. 8D) and BMRPA1.K-ras^{val12} cells (FIG. 8F) in a ring-like pattern, but did not stain the cell surface of untransformed BMRPA1 cells (FIG. 8H), indicating the presence of the 3C4-Ag on the surface membrane of only transformed cells.

TABLE 5
Immunoreactivity of selected supernatants
with intact cells by immunofluorescence.

| | Supernatants | | | | |
|---|---|---|---|---|---|
| Cells | 3D4 | 3A2 | 4AB1 | 2B5 | 3C4 |
| BMRPA1 | − | − | 3+ | +/2+ | − |
| BMRPA1.NNK | − | − | 3+ | 3+ | 3+ |
| BMRPA1.K-ras^{val12} | − | − | 3+ | +/2+ | 3+ |

* The strength of the indirect immunofluorescence staining was determined by comparing the fluorescence intensity of each sample with that seen in a parallel preparation of cells stained with serum from hyperimmunized mice (positive control, IFA = 3+) and unreactive spent hybridoma supernatant [negative control, IFA = (−)].

The other hybridoma supernatants (2B5 and 4AB1) recognizing Ags on the surface of EDTA-released intact cells, reacted with plasma membrane antigens of transformed and untransformed cells in a speckled pattern (Table 5). Interestingly, hybridoma supernatants 3D4 and 3A2 did not stain intact, EDTA-released live untransformed or transformed BMRPA1 cells. In view of the strong, persistent reactivity of 3D4 and 3A2 by Cell-EIA with BMRPA1.NNK dried cells, the absence of similar reactivity with EDTA-released intact cells by indirect immunofluorescence indicated that the 3D4 and 3A2 Ags likely have intracellular locations in transformed BMRPA1 cells.

Immunocytochemical staining of permeabilized transformed BMRPA1.NNK Cells by 3D4. To confirm a possible intracellular location of the 3D4-Ag in BMRPA1.NNK cells, immunocytochemical staining was performed on fixed, Triton-X-100 permeabilized cells. As shown in FIG. 9, the hyperimmune, positive control serum stained the whole cell body and most of the cellular components including the extended plasma membrane of spread, permeabilized BMRPA1.NNK cells (FIG. 9F). Interestingly, staining by mAb 3D4 was retained mainly in the cytoplasm and especially in the perinuclear regions of the permeabilized BMRPA1.NNK (FIG. 9E) and BMRPA1.K-ras^{val12} cells, with particularly strong staining in actively dividing cells. In contrast, mAb 3D4 did not react with permeabilized but untransformed BMRPA1 cells (FIG. 9C), whose monolayer epithelial appearance on glass slides can be nicely seen after staining with immune mouse serum raised against these cells (FIG. 9D). Most importantly, mAb 3D4 does not react with the different cell types present in normal rat pancreatic tissue, including duct, acinar and islet cells (FIG. 9A), suggesting that 3D4-Ag is a transformation associated antigen.

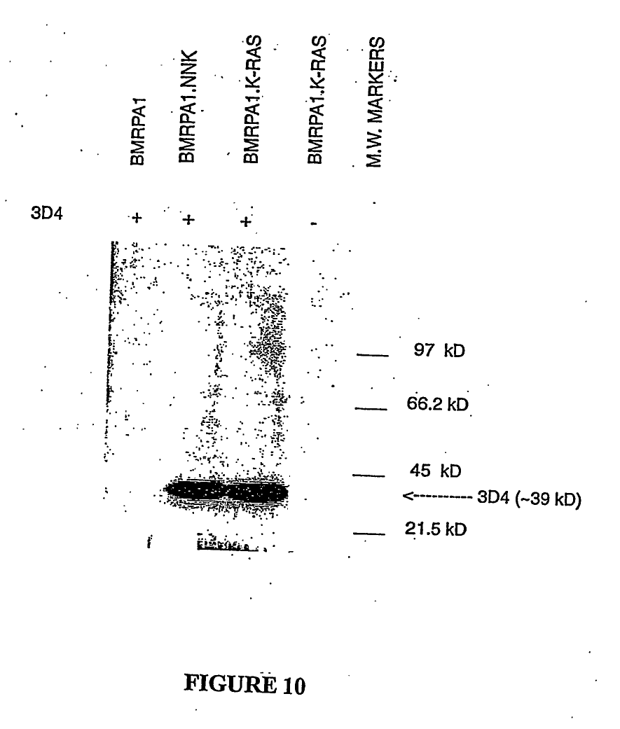

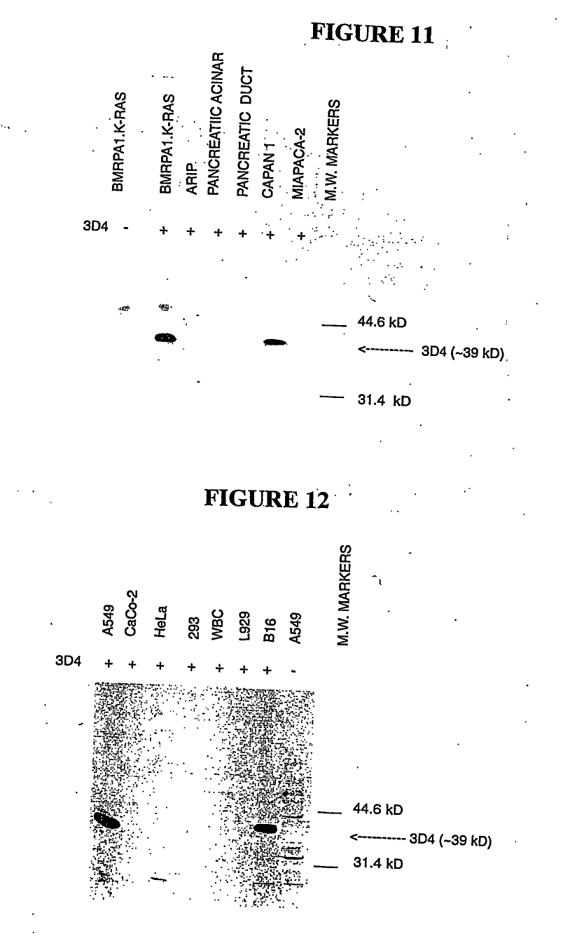

3D4-Ag is a 41.2 kD rodent and human cancer associated antigen. Western blot staining with mAb 3D4 showed a single band of ˜41.2 kD in K-Ras and NNK-transformed BMRPA1 cells, but not in untransformed BMRPA1 cells (FIG. 10). Remarkably, strong 3D4-Ag expression was also seen in human pancreatic cancer cells CAPAN1 (FIG. 11, lane 6) and CAPAN2 (not shown), as a band of molecular weight similar to the one observed in BMRPA1.K-ras^{val12} cells (FIG. 11, lane 2). The 3D4-Ag was not found in cell lysates derived from untransformed human acinar (FIG. 11, lane 4) and ductal cells (FIG. 11, lane 5). In addition, no 3D4-Ag expression was observed in ARIP (FIG. 5, lane 3), a cell line that was derived from a primary cultivation of an exocrine rat pancreatic tumor. It is important to note that ARIP cells, which are derived from a rat pancreatic tumor, display normal cell behavior and grown as a monolayer with cobblestone appearance and do not produce tumors in nude mice.

The expression of 3D4-Ag in cells from human lung cancer (A549), transformed primary embryonal kidney carcinoma (293), cervix epitheloid (HeLa), colon adenocarcinoma (CaCo-2), normal human white blood cells (WBC), mouse fibroblast (L929), and mouse melanoma cells (B16) was also examined by Western blot analysis (FIG. 12). Strong 3D4-Ag expression was observed only in A549 human lung cancer and B16 mouse melanoma cells (FIG. 12, lanes 1,7). There was no expression of 3D4 in the rest of the human carcinoma cell lines, L929 mouse fibroblast (FIG. 12) and E49 rat brain capillary endothelial cells (not shown). 3D4-Ag was not detected in normal human white blood cells (FIG. 12, lane 5), and primary human umbilical cord endothelial cells HUVEC (not shown). These results indicate 3D4-Ag is a cancer associated antigen whose epitope and molecular weight are conserved in mice, rats, and humans in a few selected cancer cells.

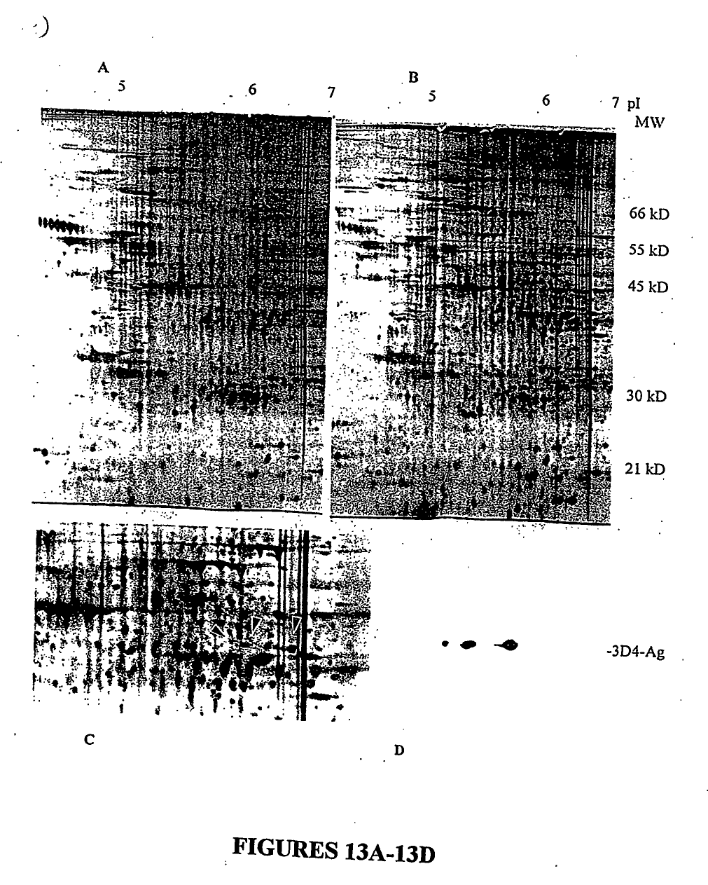

Characterization of 3D4-Ag by 2D polypeptide separation followed by silver staining and Western Blot. Two-dimensional (2D) gel electrophoresis allows the separation of thousands of polypeptides from total cell lysates according to molecular weight and isoelectric point (O'Farrell, 1975). Technological advances continue to increase the power of the 2D separation techniques by allowing larger protein amounts to be separated, making the results more reproducible, and improving both the detection methods and 2D pattern interpretation (Bauw et al., 1989; Kovarova et al., 1994). To better characterize the 3D4-Ag, 100 μg of total cell lysate protein were separated according to pI in the first dimension on a 3-10 pH gradient, followed by separation according to MW in the second dimension by Duracyl gel electrophoresis. Silver staining of gels containing 2D separated polypeptides from NNK-transformed and untransformed BMRPA1 cells showed reproducible 2D separations and polypeptide profiles (FIGS. 13 A and 13B). Silver staining of the 2D separated polypeptides from NNK-transformed and untransformed cells revealed that most polypeptides are expressed at similar levels in both untransformed and NNK-transformed cells. Nevertheless, both quantitative and qualitative polypeptide expression differences could be clearly seen between BMRPA1 and BMRPA1.NNK cells.

Transfer of the separated polypeptides from unstained gels to nitrocellulose membranes followed by Western blot analysis with the mAb 3D4 identified the 3D4-Ag as a polypeptide with three charge variants in both rat (pI˜6.24+/−0.25, 6.30+/−0.20, and 6.48 +/−0.25), and human (pI˜6.6, 6.7, and 6.9) pancreatic cancer cell lines. The polypeptide staining of the same membrane with Rev-Pro and Amido Black showed polypeptide patterns that were also detected with the more sensitive silver staining of polypeptides from gels run in parallel, helping to establish the position of the 3D4-Ag relative to the other proteins in the total cell lysate (FIG. 13D, 13C). The location of easily recognizable major proteins like actin (at 43 kD), and the molecular weight standards used (both 2D and 1D) helped to establish a molecular weight of ˜41.2 kD for the 3D4-Ag in both human and rat cells.

### REFERENCES

Aisenberg, A. C., and Davis, C. (1968). The thymus and recovery from cyclophosphamide-induced tolerance to sheep erythrocytes. J. Exp. Med. 128(1), 35-46.
Aisenberg, A. C. (1967). Studies on cyclophosphamide-induced tolerance to sheep erythrocytes. J. Exp. Med. 125, 833-845.
Alanen, K. A., Joensuu, H., Klemi, P. J., and Nevalainen, T. J., (1990). Clinical Significance of nuclear DNA content in pancreatic carcinoma. J. Path. 160, 313-320.
Amsterdam, A., and Jamieson J. D., (1974). Studies on dispersed pancreatic exocrine cells. I. Dissociation techniques and morphologic characteristics of separated cells. J. Cell Biol. 63, 1037-1056.
Appert H. (1990). Composition and Production of Pancreatic Tumor Related Antigens. Int. J. Pancreatol. 7(1-3), 13-23.
Audisio R. A., Veronesi P., Maisonneuve P., Chiappa, A., Andreoni, B., Bombardieri, E., and Geraghty, J. G. (1996). Clinical relevance of serological markers in the detection and follow-up of pancreatic adenocarcinoma. Surg. Oncol. 5, 49-63.
Bao, L.-Y, Thelmo, W. L., Somnay, S., Madahar, C., and Michl, J. (1994). Characterization of an acinar cell line, BMRPA.430, derived from adult rat pancreas. FASEB J. 8, 64A.
Barlogie, B., Raber, M. N., Schumann, J., Johnson, T. S., Drewinko, B., Swartzendruber, D. E., Gohde, W., Andreeff, M., and Freireich, E. J. (1983). Flow cytometry in clinical cancer research. Cancer Res. 43, 3982-3997.
Baskaran, K., Laconi, S., and Reddy, M. K. (1994). Transformation of hamster pancreatic duct cells by 4-(methylnitrosamino)-1-(3-pyridyl)-1-butanone (NNK), in vitro. Carcinogenesis 15(11), 2461-2466.
Bauw, G., Van Damme, J., Puype, M., Vandekerckhove, J., Gesser, B., Ratz, G. P., Lauridsen, J. B., and Celis, J. E. (1989). Protein-electroblotting and -microsequencing strategies in generating protein data bases from two-dimensional gels. Proc. Natl. Acad. Sci. USA 86, 7701-7705.
Belinsky, S. A., K. J. Nicula, W. A. Palmisano, R. Michels, G. Saccomanno, E. Gabrielson, S. B. Baylin, and J. G. Herman (1998). Aberrant methylation of p16^{INK4a} is an early event in lung cancer and a potential biomarker for early diagnosis. Proc. Natl. Acad. Sci. USA. 95: 11891-11896.
Brooks P. C., Lin J. M., French D. L., and Quigley J. P. (1993). Subtractive immunization yields monoclonal antibodies that specifically inhibit metastasis. J. Cell Biol., 122(6): 1351-9.
Chung, S. E., (1986) In vitro transformation of human epithelial cells. Biochemica and Biophysica Acta 823, 161-194.
Colburn, N. H., W. F. Vorder Bruegge, J. R. Bates, R. H. Gray, J. D. Rossen, W. H. Kelsey, and T. Shimada (1978). Correlation of anchorage-independent growth with tumorigenicity of chemically transformed mouse epidermal cells. Cancer Res., 38:624-634.
Curphey, T. J., C. I. Coon, B. K. Schaeffer, and D. S. Longnecker (1987). In vivo and in vitro genotoxicity of selected compounds toward rodent pancreas. Carcinogenesis, 8:8: 1033-7.
Friess H., Gassmann M., Buchler M. W. (1997). Adjuvant therapy of pancreatic cancer using monoclonal antibodies and immune response modifiers. Int. J. Panc. 21, 43-52.
Friess, H., Berberat, P., Schilling, M., Kunz, J., Korc, M, Buchler, M. W. (1996). Pancreatic cancer: the potential clinical relevance of alterations in growth factors and their receptors. J. Mol. Med, 74:35-42.
Gjertsen, M. K., Bakka, A., Breivik J., and Gaudernack G. (1996). Ex vivo ras peptide vaccination in patients with advanced pancreatic cancer: results of a phase I/II study. Int. J. Canc. 65(4), 450-3.
Guz, Y., Montminy, M. R., Stein, R., Leonard, J., Gainer, L. W., Wright, C. V., and Teitelman, G. (1995). Expression of murine STF-1, a putative insulin gene transcription factor, in beta cells of pancreas, duodenal epithelium and pancreatic exocrine and endocrine progenitors during ontogeny. Development 121(1), 11-18.
Hecht, S. S. (1996). Recent studies on mechanisms of bioactivation and detoxification of 4-(methylnitrosamino)-1-(3-pyridyl)-1-butanone (NNK), a tobacco-specific lung carcinogen. Critical Reviews in Toxicology 26(2), 163-181.
Hecht, S. S., and Hoffmann, D. (1991). 4-(Methylnitrosamino)-1-(3-pyridyl)-1-butanone (NNK), a nicotine-derived tobacco-specific nitrosamine and carcinognen for the lung and pancreas in humans. In Origins of Human Cancer: A Comprehensive Review, Cold Spring Harbor, N.Y., Cold Spring Harbor Laboratory, 745-755.
Hoffman, D., Brunnemann, D. D., Prokopcczyk, B., and Djordjevic, M. V. (1994). Tobacco-specific N-nitrosamines and Areca-derived N-nitrosamines: chemistry, biochemistry, carcinogenicity, and relevance to humans. J. of Tox. and Env. Health 41, 1-52.
Hruban, R. H., A. D. M. van Mansfeld, G. J. A. Offerhaus, D. H. J van Weering, D. C. Allison, S. N. Goodman, T. W. Densler, K. K. Bose, J. L. Cameron, and J. L. Bos (1993). K-ras oncogene activation in adenocaricnoma of the human pancreas. A study of 82 carcinomas using a combination of mutant-enriched polymerase chain reaction analysis and allele-specific oligonucleotide hybridization. Am. J. Pathol., 143:4682-4689.
Hruban, R. H., C. J. Yeo, and S. E. Kern (1998). Pancreatic cancer. The Basis of Human Cancer, Inc, Vogelstein B., Kinzler, K. W., McGrow-Hill Inc., N.Y., 603-605.
Huynh, R., Bradu, S. M., Akman, H. A., Carleton, S., Zheng, T., Bao L.-Y., and Michl, J. (1996). Transformation and tumor formation in BMRPA. 430 rat pancreatic acinar cells exposed in vitro to the tobacco smoke-specific carcinogen—nitrosamine NNK. Gastroenterology 110, A400.
Jones, R. T., E. A. Hudson, and J. H. Resau (1981). A review of in vitro and in vivo culture techniques for the study of pancreatic carcinogenesis. Cancer, 47:1490-1496.
Katz, M. E., and Mc Cormick, F. (1997) Signal transduction from multiple Ras effectors. Curr. Op. in Genet. and Dev., 7: 75-79, 1997.
Köhler G., and Milstein C. (1975). Continuous culture of fused cells secreting antibody of pre-destined specificity. Nature 256, 495-497.
Kovarova, H., Stulik, J., Hochstrasser, D. F., Bures, J., Melichar, B., and Jandik, P. (1994). Two-dimensional electrophoretic study of normal colon mucosa and colorectal cancer. Appl. Theoret. Electrophoresis 4, 103-106.
Laemmli, U. K. (1970). Cleavage of structural proteins during assembly of the head of bacteriophage T4. Nature 227, 680.
Lagrange, P. H., Macanese, G. B., and Miller, T. E. (1974). Potentiation of T-cell-mediated immunity by selective suppression of antibody formation with cyclophosphamide. J. Exp. Med. 139, 1529-1539.
Leget, G. A., and Czuczman, M. S. (1998). Use of rituximab, the new FDA-approved antibody. Curr. Opin. Oncol. 10(6), 548-51.
Lum, L. G. (1999). T cell-based immunotherapy of cancer: a virtual reality? CA Cancer J. Clin. 49, 74-100.
Marchand, M., van Baren, N., Weyanants, Pl., Brichard, V., Dreno, B., Tessier, M. H., Rankin, E., Parmiani, G., Arienti, F., Humblet, Y., Bourlond, A., Vanwijck, R., Lienard, D. Beauduin, M., Dietrich, P. Y., Russo, V., Derger, J., Masucci, G., Fager, E., De Greve, J., Atzpodien, J/. Brasseur, F. Coulie, P. G., van der Bruggen, P., and Boon, T. (1999). Tumor regressions observed in patients with metastatic melanoma treated with an antigenic peptide encoded by gene MAGE-3 and presented by HLA-A1. Int. J. Cancer 80(2), 219-30.
Marinova-Mutafchieva, L., Goranova, I., and Goranov, I. (1990). Positive effect of cyclophosphamide on the expression of MHC class II antigens. Meth. Find. Exp. Clin Pharmacol. 12(8), 545-549.
Metzgar, R. S., Gaillard, M. T., Levine, M. T., Levine, J. S., Tuck, F. L., Bossen, E. H., and Borowitz, M. J. (1982). Antigens of human pancreatic adenocarcinoma cells defined by murine monoclonal antibodies. Canc. Res. 42, 601-608.
Nestle, F. O., Slijagic, S., Gilliet, M., Sun, Y., Grabbe, S., Dummer, R., Burg, G., and Schadendorf, D. (1998). Vaccination of melanoma patients with peptide- or tumor lysate-pulsed dendritic cells. Nat. Med. 4 (3), 328-32.
O'Farrell, P. (1975). High resolution two-dimensional electrophoresis of proteins. J. Biol. Chem. 250(10), 4007-4021.
Old, L. J. (1981). Cancer immunology: the search for specificity-G. H. A. Clowes Memorial Lecture. Canc. Res. 41, 361-375.
Pantel, K., Djuric, Z., and Nakeff, A. (1990). Stem cell recovery from cyclophosphamide-induced myelosuppression requires the presence of CD4+ cells. Br. J. Haematology 75, 168-174.
Parsa, I., Foye, C. A., Cleary, C. M., and Hoffmann, D. (1986). Differences in metabolism and biological effects of NNK in human target cells. Banbury Rep. 23, 233-244.
Pegram, M. D., A. Lipton, D. F. Hayes, B. L. Weber, J. M. Baselga, D. Tripathy, D. Baly, Baughman, S. A., T. Twaddell, J. A. Glaspy, and Slamon, D. J. (1998). Phase II study of receptor-enhanced chemosensitivity using recombinant humanized anti-p185HER2/neu monoclonal antibody plus cisplatin in patients with HER2/neu-overexpressing metastatic breast cancer refractory to chemotherapy treatment. J. Clin. Oncol. 16(8), 2659-71.
Playfair, J. H. L. (1969). Specific tolerance to sheep erythrocytes in mouse bone marrow cells. Nature 222, 882-883.
Pytowski B., Easton T. G., Valinski J. E., Calderon, T., Sun, T., Christman, J. K., Wright, S. D., and Michl, J. (1988). A monoclonal antibody to human neutrophil-specific plasma membrane antigen. J. Exp. Med. 167, 421-439.
Rosenberg, S A, Yang J C, Schwartzentruber D J, Hwu, P., Marincola, F. M., Topalian, S. L., Restifo, N. P., Dudley, M. E., Schwarz, S. L., Wunderlich, J. R., Parkhurst, M. R., Kawakami, Y., Seipp, C.A., Einhorn, J. H., and White, D. E. (1998). Immunologic and therapeutic evaluation of a synthetic peptide vaccine for the treatment of patients with metastatic melanoma. Nature Med 4, 321-327.
Serrano, M., A. W. Lin, M. E. McCurrach, D. Beach, and S. W. Lowe (1997). Oncogenic ras provokes premature cell senescence associated with accumulation of p53 and p16INK4a. Cell, 88:5:593-602.
Shawler et al. (1997) Advances in Pharmacology 40:309-337, Academic Press.
Shen, R., Su, Z., Olsson, C. A., Goldstein, N. I., and Fisher, P. B. (1994). Surface-epitope masking: a strategy for the development of monoclonal antibodies specific for molecules expressed on the cell surface. J. Natl. Cancer. Inst. 86(2), 91-98.
Shin, S. I., V. H. Freedman, R. Risser, and R. Pollack (1975). Tumorigenicity of virus-transformed cells in Nu/Nu mice is correlated specifically with anchorage independent growth in vitro. Proc. Natl. Acad. Sci. USA 72: 11:4435-39.
Srivastava, P. K, and L. J. Old. (1988). Individually distinct transplantation antigens of chemically induced mouse tumors. Immun. Today, 9(3), 78-83.
Srivastava, P. K. (1996). Do human cancers express shared protective antigens? Or the necessity of remembrance of things past. Semin. Immun. 8, 295-302.
Staretz, M. E., and S. E. Hecht (1995). Effects of phenethyl isothiocyanate on the tissue distribution of 4-(methylnitrosamino)-(3-pyridyl)-1-butanone and metabolites in F344 rats. Cancer Res., 55:5580-88.
Streit M., R. Schmidt, R. U. Hilgenfeld, E. Thiel. and E. D. Kreuser (1996). Adhesion receptors in malignant transformation and dissemination of gastrointestinal tumors. Molecular Med., 74:253-268.
Turk, J. L., Parker, D., and Poulter, L. W. (1972). Functional aspects of the selective depletion of lymphoid tissue by cyclophosphamide. Immunology. 23, 493-501.
Van Benthem, J., V. J. Feron, W. R Leeman., J. W. G. M. Wilmer, E. Vermeulen, L. den Engelse, and Scherer (1994). Immunocytochemical identification of DNA adducts, 06-methylguanine and 7-methylguanine, in respiratory and other tissues of rat, mouse, and Syrian hamster exposed to 4-(methlynitrosamino)-1-(3-pyridyl)-1-butanone. Carcinogenesis, 15:9: 2023-2029.
Williams C. V., McLoon, S. C., and Stechmann, C. L. (1992). Subtractive immunization techniques for the production of monoclonal antibodies to rare antigens. Biotechniques 12(6), 842-847.

What is claimed is:

1. A method for redirecting the immune response of an animal towards immunologically weak or rare antigens, said method comprising:
(a) administering to the animal a first set of antigens and allowing a first and secondary immune response;
(b) administering to the animal an immunosuppressant which inhibits growth of rapidly proliferating immune cells;
(c) administering to the animal a second set of antigens which is similar or related to, but distinct from, the first set of antigens; and
(d) administering booster injections of the second set of antigens sufficient to raise the antibody titer to the second set of antigens and to cause increased immigration of plasma cells secreting antibodies to the second set of antigens into the spleen of the animal.

2. A method of producing monoclonal antibodies which react specifically with immunologically weak or rare antigens, said method comprising:
(a) administering to an animal a first set of antigens and allowing a first and secondary immune response;
(b) administering to the animal an immunosuppressant which inhibits growth of rapidly proliferating immune cells;
(c) administering to the animal a second set of antigens winch is similar or related to, but distinct from, the first set of antigens;
(d) administering booster injections of the second set of antigens sufficient to raise the antibody titer to the second set of antigens and to cause increased immigration of plasma cells secreting antibodies to the second set of antigens into the spleen of the animal;
(e) isolating splenocytes from the animal; and
(f) fusing the isolated splenocytes with myeloma cells or transformed cells capable of replicating indefinitely in culture to yield hybridomas which secrete the monoclonal antibodies that react specifically with the immunologically weak or rare antigens.

3. The method of claim 1 or 2 wherein the immunosuppressant is cyclophosphamide.

4. The method of claim 1 or 2 wherein the first set of antigens comprises untransformed cells and the second set of antigens comprises cells derived therefrom which are neoplastically transformed.

5. The method of claim 1 or 2 wherein the second set of antigens comprise antigens in both native and denatured form.

6. The method of claim 4 wherein the first set of antigens comprises BMRPA1 (BMPRA.430) cells and the second set of antigens comprises BMRPA1.NNK cells.

7. The method of claim 4 wherein the first set of antigens comprises BMRPA1 (BMPRA.430) cells and the second set of antigens comprises TUC3 (BMRPA1.K-ras^{Val12}) cells.

8. The method of claim 4 wherein the second set of antigens comprises a tumor associated antigen or a tumor specific antigen.

9. The method of claim 8 wherein the cancer associated antigen is a pancreatic cancer associated antigen.

10. The method of claim 8 wherein the tumor associated antigen is a pancreatic tumor associated antigen.

11. A culture medium capable of maintaining BMRPA1 cells in a differentiated state wherein the culture medium comprises: about 0.02 M glutamine, about 0.01 to about 0.1 M HEPES-Buffer, bovine insulin dissolved in acetic acid in a range of from about 0.001 to about 0.01 mg/mL acetic acid/L of medium), about 1 to about 8×10⁻⁷ M ZnSO₄, about 1 to about 8×10⁻¹⁰ M NiSO₄ 6H₂O, 5×10⁻⁷ to about 5×10⁻⁶ CuSO₄, about 5×10⁻⁷ to about 5×10⁻⁶ FeSO₄, about 5×10⁻⁷ to about 5×10⁻⁶ M MnSO₄, about 5×10⁻⁷ to about 5×10⁻⁶ M (NH₄)₆Mn₇O₂₄, about 0.3 to about 0.7 mg/L medium Na₂SeO₃, about 1×10⁻¹⁰ to about 8×10⁻¹⁰ M SnCl₂ 2H₂O and about 5×10⁻⁴ to about 5×10⁻⁵ M carbamyl choline, wherein said medium has a pH adjusted in the range of from about 6.8 to 7.4.

12. A monoclonal antibody produced by the method of claim 2.

13. Transformed BMRPA1 (BMPRA.430) cells exposed to 1 μg NNK/ml culture medium from about 12 to about 24 hours.

14. The cell line BMRPA1.NNK, derived from the cells of claim 13.

15. The cell line TUNNK, derived from a tumor of a mouse injected with is BMRPA1.NNK cells.

16. A cancer associated antigen 3D4-Ag in substantially pure from characterized by:
a molecular weight of about 41.2 kD as determined by SDS-PAGE;
a pI on isoelectrofocusing of about 5.9 to about 6.9; and,
detectable in BMRPA1.NNK cells, BMRPA1.TUC3 cells, BMRPA1.TUNNK cells, human pancreatic cancer cell line CAPAN1, CAPAN2, A549 human lung cancer cells, and B16 mouse melanoma cells.

17. An antibody having specific binding specificity to cancer associated antigen 3D4-Ag wherein said antigen is characterized by:
a molecular weight of about 41.2 kD as determined by SDS-PAGE;
a pI on isoelectrofocusing of about 5.9 to about 6.9; and,
detectable in BMRPA1.NNK cells, BMRPA1.TUC3 cells, BMRPA1.TUNNK cells, human pancreatic cancer cell line CAPN1, CAPAN2, A549 human lung cancer cells, and B16 mouse melanoma cells.

18. The antibody of claim 17 which is a monoclonal antibody.

19. A murine hybridoma cell line which produces a monoclonal antibody specifically immunoreactive with the 3D4-Ag of claim 16.

20. A monoclonal antibody mAb3D4, secreted by the hybridoma of claim 19.

21. A hybridoma produced by the method of claim 6 wherein the hybridoma produces an antibody which binds to antigens on the surface of BMRPA1 and BMRPA1.NNK cells.

22. An antibody produced by the hybridoma of claim 21 wherein said antibody is mAb4AB1 or mAb2B5.

23. A hybridoma produced by the method of claim 6 wherein the hybridoma produces an antibody which binds to antigens of BMRPA1.NNK cells but not untransformed BMRPA1 cells.

24. An antibody produced by the hybridoma of claim 23 wherein the antibody is mAb3A2.
